SEQUENCE LISTING (26 sequences)

SEQ ID NO: 1 - protein, 106 aa, Mus musculus
QSPASLAVSLGQRATISCRASESVDSFGNSFMHWYQQKAGQPPKSSIYRAANLESGIPARFSGSGSRTDFTLTINPVEADDVATYFCQQSYEDPWTFGGGTKLGNQ

SEQ ID NO: 2 - protein, 105 aa, Mus musculus
LVKPGGSLKLSCVASGFTFSSYAMSWVRQTPEKRLEWVASISSGGTTYYPDNVKGRFTISRDNARNILYLQMSSLRSEDTAMYYCARGYYDYHYWGQGTTLTVSS

SEQ ID NO: 3 - protein, 106 aa, Mus musculus
QSPASLAVSLGQRATISCRASESVDSYVNSFLHWYQQKPGQPPKLLIYRASNLQSGIPARFSGSGSRTDFTLTINPVEADDVATYCCQQSNEDPTTFGGGTKLEIK

SEQ ID NO: 4 - protein, 112 aa, Mus musculus
LQQSGPGLVKPSQSLSLTCTVTGYSITSDSAWNWIRQFPGNRLEWMGYISYSGSTSYNPSLKSRISITRDTSKNQFFLQLNSVTTEDTATYYCVRGLRFAYWGQGTLVTVSA

SEQ ID NO: 5 - protein, 101 aa, Mus musculus
QTPSSLSASLGDRVTISCRASQDIKNYLNWYQQPGGTVKVLIYYTSRLHSGVPSRFSGSGSGTDYSLTISNLEQEDFATYFCQRGDTLPWTFGGGTKLEIK

SEQ ID NO: 6 - protein, 116 aa, Mus musculus
LESGPGLVAPSQSLSITCTVSGFSLTNYDISWIRQPPGKGLEWLGVIWTSGGTNYNSAFMSRLSITKDNSESQVFLKMNGLQTDDTGIYYCVRGDGNFYLYNFDYWGQGTTLTVSS

SEQ ID NO: 7 - protein, 15 aa, Mus musculus
RASESVDSFGNSFMH

SEQ ID NO: 8 - protein, 7 aa, Mus musculus
RAANLES

SEQ ID NO: 9 - protein, 9 aa, Mus musculus
QQSYEDPWT

SEQ ID NO: 10 - protein, 5 aa, Mus musculus
SYAMS

SEQ ID NO: 11 - protein, 16 aa, Mus musculus
SISSGGTTYYPDNVKG

SEQ ID NO: 12 - protein, 7 aa, Mus musculus
GYYDYHY

SEQ ID NO: 13 - protein, 15 aa, Mus musculus
RASESVDSYVNSFLH

SEQ ID NO: 14 - protein, 7 aa, Mus musculus
RASNLQS

SEQ ID NO: 15 - protein, 9 aa, Mus musculus
QQSNEDPTT

SEQ ID NO: 16 - protein, 6 aa, Mus musculus
SDSAWN

SEQ ID NO: 17 - protein, 16 aa, Mus musculus
YISYSGSTSYNPSLKS

SEQ ID NO: 18 - protein, 6 aa, Mus musculus
GLRFAY

SEQ ID NO: 19 - protein, 11 aa, Mus musculus
RASQDIKNYLN

SEQ ID NO: 20 - protein, 7 aa, Mus musculus
YTSRLHS

SEQ ID NO: 21 - protein, 9 aa, Mus musculus
QRGDTLPWT

SEQ ID NO: 22 - protein, 5 aa, Mus musculus
NYDIS

SEQ ID NO: 23 - protein, 16 aa, Mus musculus
VIWTSGGTNYNSAFMS

SEQ ID NO: 24 - protein, 12 aa, Mus musculus
GDGNFYLYNFDY

SEQ ID NO: 25 - protein, 238 aa, Artificial Sequence
  IC14 VL domain
METDTILLWVLLLWVPGSTGDIVLTQSPASLAVSLGQRATISCRASESVDSYVNSFLHWYQQKPGQPPKLLIYRASNLQSGIPARFSGSGSRTDFTLTINPVEADDVATYYCQQSNEDPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 460 aa, Artificial Sequence
  IC14 VH domain
MKVLSLLYLLTAIPGILSDVQLQQSGPGLVKPSQSLSLTCTVTGYSITSDSAWNWIRQFPGNRLEWMGYISYSGSTSYNPSLKSRISITRDTSKNQFFLQLNSVTTEDTATYYCVRGLRFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2021/0231686 A1
(43) Pub. Date: Jul. 29, 2021

(54) METHODS FOR PROGNOSIS AND MANAGEMENT OF DISEASE

(71) Applicant: The Methodist Hospital, Houston, TX (US)

(72) Inventors: Stanley H. Appel, Houston, TX (US); David R. BEERS, Houston, TX (US)

(73) Assignee: The Methodist Hospital, Houston, TX (US)

(21) Appl. No.: 17/054,131

(22) PCT Filed: May 10, 2019
(86) PCT No.: PCT/US2019/031858

§ 371 Date: Nov. 9, 2020

Related U.S. Application Data
(60) Provisional application No. 62/669,915, filed on May 10, 2018.

(51) Int. Cl. G01N 33/68

(52) U.S. Cl. CPC G01N 33/6896; G01N 2800/52; G01N 2333/70596; G01N 2800/28

(57) ABSTRACT

The present disclosure relates generally to methods for the prognosis and management of amyotrophic lateral sclerosis (ALS), as well as associated compositions, kits, solid supports and uses.

### FIELD OF THE DISCLOSURE

The present disclosure relates generally to methods for the prognosis and management of amyotrophic lateral sclerosis (ALS), as well as associated compositions, kits, solid supports and uses.

### RELATED APPLICATION

This application claims priority to U.S. Provisional Application No. 62/669,915 entitled “Methods for prognosis and management of disease” filed 10 May 2018, the contents of which are incorporated herein by reference in their entirety.

### INCORPORATION BY REFERENCE OF SEQUENCE LISTING

The present application is being filed along with a Sequence Listing in electronic format. The Sequence Listing is provided as a file entitled 35524033 Implicit ALS PCT_ST25.TXT, created May 7, 2019, which is 15,144 bytes in size. The information in the electronic format of the Sequence Listing is incorporated by reference in its entirety.

### BACKGROUND OF THE DISCLOSURE

Amyotrophic lateral sclerosis (ALS), also known as Lou Gehrig's disease, is the most common, devastating, and invariably fatal adult degenerative motor neuron disease that selectively destroys upper and lower motor neurons. About 10% of ALS patients have a positive family history with disease usually inherited as dominant traits, while 90% are sporadic without a family history, reflecting a complex interaction between genetic susceptibility and the environment. The ALS “syndrome” has a clinically heterogeneous presentation and course, with survival times ranging from a few months to several decades, but typically ranging from 3 to 5 years from the onset of disease symptoms. Survival is associated with several factors such as clinical phenotype, age at onset, gender, early presence of respiratory failure, and weight loss (Lunetta C. et al., JAMA Neurol. 2017; 74(6):660-667). The pathological process in ALS is now recognized to extend beyond the motor system, and includes cognitive impairments as-well-as dysregulated central and peripheral immune systems. Unfortunately, minimal effective treatment is currently available to modify the pathobiology of this disease; palliative care and symptomatic treatment are essential components in the management of these patients.

ALS is characterized by heterogeneity in the region of onset, rate of progression, patterns of disease spread, and relative burden of upper motor neuron (UMN), lower motor neuron (LMN), and cognitive pathology. This phenotypic variability in ALS complicates measurement of disease progression. With the dawning era of targeted therapeutics in ALS, accurate measurement of disease burden and rate of progression remains a critical priority to facilitate efficient clinical trial design and to enable further insights into disease pathogenesis for the development of therapeutics for ALS.

Understanding the rate of progression of ALS in a subject can assist in developing an appropriate management or treatment plan, in additional to being more generally useful to the subject for planning other aspects of their lives. With most diseases, a more rapid rate of progression indicates the need for a more aggressive treatment with drugs versus physical therapy or nutritional support; a more potent drug; or perhaps the same drug delivered at a higher dose or at shorter intervals between dosing cycles. In some instances, certain therapies may be more effective in rapid versus slow progressing diseases. The absence of a prognostic that predicts the rate of progression at the time of sampling is therefore a barrier to the personalization of an ALS/MND treatment regimen to optimize the outcome for an individual patient. Therefore, there remains a need to identify methods for accurately predicting or determining the progression rate of a subject, such as whether a subject with ALS has slow or rapid progressing disease.

### SUMMARY OF THE DISCLOSURE

The present disclosure is predicated, at least in part, on the identification of ALS progression biomarkers that correlate with the rate of progression of ALS in a subject. As demonstrated herein, the level of one or more ALS progression biomarkers in a biological sample of a subject with rapid progressing ALS is typically increased (or elevated) compared to the level of the same biomarker in a biological sample of a subject with slow progressing ALS and compared to a healthy subject. Conversely, the level of one or more ALS progression biomarkers in a biological sample of a subject with slow progressing ALS is typically decreased (or reduced) compared to the level of the same biomarker in a biological sample of a subject with rapid progressing ALS. Accordingly, determining the level of the one or more ALS progression biomarkers in a biological sample of a subject with ALS can be used to determine the rate of progression of ALS. In particular embodiments, determining the level of the one or more ALS progression biomarkers in a biological sample of a subject is used to determine whether that subject is likely to have slow progressing ALS or rapid progressing ALS. Exemplary of such ALS progression biomarkers is soluble CD14 (sCD14) and lipopolysaccharide binding protein (LBP).

An earlier understanding of whether a subject with ALS is likely to have rapid or slow disease progression, which the present disclosure provides the means to do, has clear benefits and advantages. For a subject diagnosed with ALS, knowing whether they are likely to have rapid or slow disease progression can assist in making the appropriate lifestyle and financial decisions. Moreover, understanding the likely disease progression rate may impact treatment regimens. For example, respiratory function is one of the major factors dictating survival, and having a marker of fast progression may prompt earlier use of non-invasive ventilation (NIV). Like respiratory function, timing of insertion of a feeding tube is critical and a factor that impacts life expectancy. Once a subject with ALS has progressed to the point where their respiratory function has significantly deteriorated then anaesthesiologists can be reluctant to assist in surgery. As a result, it is desirable that subjects understand their disease progression so that a feeding tube can be inserted early if there is any concern about rapid progression. In other examples, subjects that have rapid progressing ALS may be identified and selected for inclusion in clinical trials assessing the efficacy of new therapies. The inclusion of such subjects in clinical trials is desirable because the shorter time period for disease progression can yield more rapid evidence of efficacy of the therapy.

Accordingly, in one aspect, provided is a method for assessing the rate of progression of ALS in a subject, the method comprising: (a) determining the level of a biomarker in a biological sample obtained from a subject with ALS, wherein the biomarker is soluble CD14 (sCD14) or lipopolysaccharide binding protein (LBP); and (b) determining the likely rate of progression of ALS in the subject based on the level of the biomarker in the biological sample, e.g. relative to a suitable reference level. In particular embodiments, a determination of the rate of progression of ALS facilitates a determination of whether a subject with ALS is likely to have rapid or slow progressing ALS. In such instances, the method comprises: (a) determining the level of a biomarker in a biological sample obtained from a subject with ALS, wherein the biomarker is soluble CD14 (sCD14) or lipopolysaccharide binding protein (LBP); and (b) determining whether the subject is likely to have rapid or slow progressing ALS based on the level of the biomarker in the biological sample relative to a suitable reference level.

Thus, provided is a method for determining whether a subject with ALS is likely to have rapid or slow progressing ALS, the method comprising: (a) determining the level of a biomarker in a biological sample obtained from a subject with ALS, wherein the biomarker is soluble CD14 (sCD14) or lipopolysaccharide binding protein (LBP); and (b) determining whether the subject is likely to have rapid or slow progressing ALS based on the level of the biomarker in the biological sample relative to a suitable reference level. For example, where the reference level is representative of a healthy subject and/or a subject known to have slow progressing ALS, an increase in the level of the biomarker relative to the reference level can indicate that the subject is likely to have rapid progressing ALS. In another example, where the reference level is representative of a subject known to have rapid progressing ALS, a similar level of the biomarker relative to the reference level can indicate that the subject is likely to have rapid progressing ALS. In a further example, where the reference level is representative of a healthy subject and/or a subject known to have slow progressing ALS, a similar level of the biomarker relative to the reference level can indicate that the subject is likely to have slow progressing ALS. In a still further example, where the reference level is representative of a subject known to have rapid progressing ALS, a decrease in the level of the biomarker relative to the reference level can indicate that the subject is likely to have slow progressing ALS. In particular embodiments, the reference level is a threshold level above which the subject is likely to have rapid progressing ALS, and below which the subject is likely to have slow progressing ALS.

In specific examples of the methods described above and herein, the method comprises determining the level of both sCD14 and LBP. Typically, the biological sample is selected from the group consisting of blood, plasma, serum, urine and cerebrospinal fluid (CSF). In some embodiments, the method further comprises measuring the level of at least one other biomarker in the biological sample. In one example, the at least one other biomarker is selected from the group consisting of CRP, MIF, sTNFRI and/or sTNFRII. For example, an increase in the level of CRP, sTNFRI and/or sTNFRII relative to a reference level that is representative of a healthy subject or a subject with slow progressing ALS can indicate that the subject has rapid progressing ALS.

In some embodiments of the methods of the present disclosure, an additional step of obtaining the biological sample prior to measuring the level of the biomarker is performed. In other embodiments, the methods include exposing the subject to a treatment regimen for treating ALS.

Also provided is a kit for determining the level of a biomarker in a subject with ALS, wherein the kit comprises an antigen-binding molecule specific for the biomarker which allows for measuring the level of the biomarker in a biological sample, wherein the biomarker is sCD14 or LBP. In some embodiments, the kit comprises an antigen-binding molecule specific for sCD14 and an antigen-binding molecule specific for LBP, which allow for measuring the level of sCD14 and LBP in a biological sample. In further embodiments, the kit also comprises an antigen-binding molecule specific for at least one other biomarker selected from among CRP, MIF, sTNFRI, sTNFRII, NFL, pNfH, p75NTR^{ECD}, miR-206, miR-143-3p, and miR-374b-5p. The kit may also contain one or more detection agents, and/or instructional material for measuring the level of the biomarker(s).

In another aspect, the disclosure provides a solid support, comprising an antigen-binding molecule specific for a biomarker, wherein the biomarker is sCD14 or LBP. In some examples, the support comprises an antigen-binding molecule specific for sCD14 and an antigen-binding molecule specific for LBP. In further examples, the solid support additionally comprises an antigen-binding molecule specific for at least one other biomarker selected from among CRP, MIF, sTNFRI, sTNFRII, NFL, pNfH, p75NTR^{ECD}, miR-206, miR-143-3p, and/or miR-374b-5p. In some embodiments, the solid support is selected from among a multiwell plate, a slide, a chip or a plurality of beads.

In a further aspect, provided is a method of stratifying a subject to a treatment for ALS, the method comprising (a) determining whether or not a subject is likely to have rapid or slow progressing ALS, or assessing the rate of progression of ALS in a subject, in accordance with the methods described above and herein; and (b) determining an optimized treatment regime suitable for the subject based on whether the subject is likely to have rapid or slow progressing ALS, or based on the rate of progression of ALS in the subject. Such methods for stratifying subjects may also further involve exposing the subject to the optimized treatment regimen.

The present disclosure also provides a method for treating a subject that is likely to have rapid progressing ALS, the method comprising (a) selecting a subject that is likely to have rapid progressing ALS on the basis of the level of a biomarker in a biological sample of the subject, wherein the biomarker is sCD14 or LBP; and (b) exposing the subject to a treatment regimen optimized for treating rapid or progressing ALS. The biological sample may be, for example, blood, plasma, serum, urine or CSF. In some embodiments, step (a) comprises selecting a subject that is likely to have rapid progressing ALS on the basis of the levels of sCD14 and LBP in the biological sample of the subject. In further embodiments of this method of treatment, prior to selecting the subject, the method involves determining whether the subject is likely to have rapid progressing ALS in accordance with the methods described above and herein.

In some embodiments, the treatment regimen comprises the administration of an anti-neurodegenerative agent, such as, for example, riluzole, edaravone, a CD14 antagonist, GM604, masitinib, a complement pathway inhibitor (e.g. C5a inhibitor, such as PMX205 or eculizumab), or an agent that blocks the interaction between CD40 and CD40 ligand (e.g. an antibody that binds specifically to CD40 and/or CD40 ligand, such as the AT-1502 antibody). In particular embodiments, the CD14 antagonist is a CD14 antagonist antibody, such as one selected from:

(1) an antibody comprising:

a VL domain that comprises, consists or consists essentially of the sequence:

[SEQ ID NO: 1]
QSPASLAVSLGQRATISCRASESVDSFGNSFMHWYQQKAGQPPKSSIYRA
ANLESGIPARFSGS GSRTDFTLTINPVEADDVATYFCQQSYEDPWTFGG
GTKLGNQ (3C10 VL);

and

a VH domain that comprises, consists or consists essentially of the sequence:

[SEQ ID NO: 2]
LVKPGGSLKLSCVASGFTFSSYAMSWVRQTPEKRLEWVASISSGGTTYYP
DNVKGRFTISRDNARNILYLQMSSLRSEDTAMYYCARGYYDYHYWGQGTT
LTVSS (3C10 VH);

(2) an antibody comprising:

a VL domain that comprises, consists or consists essentially of the sequence:

[SEQ ID NO: 3]
QSPASLAVSLGQRATISCRASESVDSYVNSFLHWYQQKPGQPPKLLIYRA
SNLQSGIPARFSGSGSRTDFTLTINPVEADDVATYCCQQSNEDPTTFGGG
TKLEIK (28C5 VL);

and

a VH domain that comprises, consists or consists essentially of the sequence:

[SEQ ID NO: 4]
LQQSGPGLVKPSQSLSLTCTVTGYSITSDSAWNWIRQFPGNRLEWMGYIS
YSGSTSYNPSLKSRISITRDTSKNQFFLQLNSVTTEDTATYYCVRGLRFA
YWGQGTLVTVSA (28C5 VH);

and

(3) an antibody comprising:

a VL domain that comprises, consists or consists essentially of the sequence: QTPSSLSASLGDRVTISCRASQDIKNYLNWYQQPGGTVKVLIYYTSRLHSGVPSRFSGSGSGTDY SLTISNLEQEDFATYFCQRGDTLPWTFGGGTKLEIK [SEQ ID NO: 5] (18E12 VL); and

a VH domain that comprises, consists or consists essentially of the sequence:

[SEQ ID NO: 6]
LESGPGLVAPSQSLSITCTVSGFSLTNYDISWIRQPPGKGLEWLGVIWTS
GGTNYNSAFMSRLSITKDNSESQVFLKMNGLQTDDTGIYYCVRGDGNFYL
YNFDYWGQGTTLTVSS (18E12 VH).

In further embodiments, the treatment regimen comprises exposing the subject to non-invasive ventilation, such as Average Volume Assured Pressure Support (AVAPS), Continuous Positive Airway Pressure (CPAP) and/or Bilevel Positive Airway Pressure (BiPAP). In particular examples, the subject that is likely to have rapid progressing ALS is exposed to non-invasive ventilation earlier than if the subject was likely to have slow progressing ALS.

The present disclosure also provides a method for treating a subject that is likely to have slow progressing ALS, the method comprising (a) selecting a subject that is likely to have slow progressing ALS on the basis of the level of a biomarker in a biological sample of the subject, wherein the biomarker is sCD14 or LBP; and (b) exposing the subject to a treatment regimen optimized for treating slow progressing ALS. The biological sample may be, for example, blood, plasma, serum, urine or CSF. In some instances, step (a) comprises selecting a subject that is likely to have slow progressing ALS on the basis of the levels of sCD14 and LBP in the biological sample of the subject. In particular embodiments of the method for treating a subject that is likely to have slow progressing ALS includes, the method includes, prior to selecting the subject, a step of determining whether the subject is likely to have slow progressing ALS in accordance with the methods described above and herein.

In some examples, the treatment regimen does not comprise the administration of an anti-neurodegenerative agent, while in other examples, the treatment regimen comprises the administration of an anti-neurodegenerative agent, such as, for example, riluzole, edaravone, a CD14 antagonist, GM604, masitinib, a complement pathway inhibitor (e.g. C5a inhibitor, such as PMX205 or eculizumab), or an agent that blocks the interaction between CD40 and CD40 ligand (e.g. an antibody that binds specifically to CD40 and/or CD40 ligand, such as the AT-1502 antibody). In particular embodiments, the CD14 antagonist is a CD14 antagonist antibody, such as one selected from:

(1) an antibody comprising:

a VL domain that comprises, consists or consists essentially of the sequence:

[SEQ ID NO: 1]
QSPASLAVSLGQRATISCRASESVDSFGNSFMHWYQQKAGQPPKSSIYRA
ANLESGIPARFSGSGSRTDFTLTINPVEADDVATYFCQQSYEDPWTFGGG
TKLGNQ (3C10 VL);

and

a VH domain that comprises, consists or consists essentially of the sequence:

[SEQ ID NO: 2]
LVKPGGSLKLSCVASGFTFSSYAMSWVRQTPEKRLEWVASISSGGTTYYP
DNVKGRFTISRDNARNILYLQMSSLRSEDTAMYYCARGYYDYHYWGQGTT
LTVSS (3C10 VH);

(2) an antibody comprising:

a VL domain that comprises, consists or consists essentially of the sequence:

[SEQ ID NO: 3]
QSPASLAVSLGQRATISCRASESVDSYVNSFLHWYQQKPGQPPKLLIYRA
SNLQSGIPARFSGSGSRTDFTLTINPVEADDVATYCCQQSNEDPTTFGGG
TKLEIK (28C5 VL);

and

a VH domain that comprises, consists or consists essentially of the sequence:

[SEQ ID NO: 4]
LQQSGPGLVKPSQSLSLTCTVTGYSITSDSAWNWIRQFPGNRLEWMGYIS
YSGSTSYNPSLKSRISITRDTSKNQFFLQLNSVTTEDTATYYCVRGLRFA
YWGQGTLVTVSA (28C5 VH);

and

(3) an antibody comprising:

a VL domain that comprises, consists or consists essentially of the sequence:

[SEQ ID NO: 5]
QTPSSLSASLGDRVTISCRASQDIKNYLNWYQQPGGTVKVLIYYTSRLHS
GVPSRFSGSGSGTDYSLTISNLEQEDFATYFCQRGDTLPWTFGGGTKLEI
K (18E12 VL);

and

a VH domain that comprises, consists or consists essentially of the sequence:

[SEQ ID NO: 6]
LESGPGLVAPSQSLSITCTVSGFSLTNYDISWIRQPPGKGLEWLGVIWTS
GGTNYNSAFMSRLSITKDNSESQVFLKMNGLQTDDTGIYYCVRGDGNFYL
YNFDYWGQGTTLTVSS (18E12 VH).

Also contemplated is the use of an antigen-binding molecule specific for a biomarker in the preparation of a kit for determining whether a subject with ALS is likely to have rapid or slow progressing ALS, wherein the biomarker is sCD14 or LBP. In some examples, the use is of the combination of an antigen-binding molecule specific for sCD14 and an antigen-binding molecule specific for LBP in the preparation of a kit for determining whether a subject with ALS is likely to have rapid or slow progressing ALS. In further embodiments, an antigen-binding molecule specific for at least one other biomarker (e.g. CRP, MIF, sTNFRI, sTNFRII, NFL, pNfH, p75NTRECD, miR-206, miR-143-3p, or miR-374b-5p) is also used in the preparation of the kit.

### BRIEF DESCRIPTION OF THE DRAWINGS

FIG. 1 shows the results of flow cytometry analysis of peripheral blood mononuclear cells (PBMC) and isolated pan monocytes from subjects with ALS (separated into fast and slow progressing ALS) and healthy volunteers (control or NC). Cells were stained with anti-human CD14-V450 antibody and anti-human CD16-FITC and subjected to flow cytometry to assess monocyte populations. In the following graphical representations, white bars are CD14+/CD16− monocytes; grey bars are CD14+/CD16+ monocytes; and black bars are CD14low/CD16+ monocytes. (A) Monocyte subpopulations in PBMC; (B) Monocyte subpopulations in isolated pan monocytes; (C) CD14 expression on monocyte subpopulations in PBMC; and (D) CD14 expression on monocyte subpopulations in isolated pan monocytes.

FIG. 2 provides graphs showing the correlations between the percentage of CD14^{-/low}/CD16+ monocytes and (A) disease burden or (B) disease progression.

FIG. 3 shows the results of flow cytometry analysis of peripheral PBMC and isolated pan monocytes from subjects with ALS (separated into fast and slow progressing ALS) and healthy volunteers (NC). Cells were stained with anti-human CD14-V450 antibody, anti-human CD16-FITC and anti-human TIM3-PE and subjected to flow cytometry to assess monocyte populations. (A) Monocyte subpopulations in PBMC; (B) Monocyte subpopulations in isolated pan monocytes; (C) Correlation between CD14^{-/low}/CD16⁺/TIM-3⁺ monocytes and disease progression rate; and (D) Correlation between CD14^{-/low}/CD16⁺/TIM-3⁺ monocytes and disease progression rate.

FIG. 4 provides levels of sCD14, as measured by ELISA, in biological samples from healthy volunteers (healthy controls) and subjects with ALS. (A) serum sCD14 levels in healthy controls and ALS patients; (B) serum sCD14 levels in healthy controls and ALS patients with slow or rapid progressing disease; (C) sCD14 in the CSF of healthy volunteers and ALS patients; (D) sCD14 in the CSF of healthy controls and ALS patients with slow or rapid progressing disease; (E) correlation between serum and CSF sCD14 levels; (F) sCD14 in the urine of healthy controls and ALS patients; (G) sCD14 in the urine of healthy controls and ALS patients with slow or rapid progressing disease.

FIG. 5 provides levels of CD14 mRNA, as measured by qRT-PCR of mRNA isolated from PBMC, in healthy volunteers (healthy controls) and subjects with ALS. (A) CD14 mRNA in PBMC of healthy controls and ALS patients; (B) CD14 mRNA in PBMC of healthy controls and ALS patients with slow or rapid progressing disease.

FIG. 6 shows the levels of sCD14, as measured by ELISA, in biological samples from healthy volunteers (healthy controls or controls), subjects with ALS and subjects with other neurological conditions. (A) serum sCD14 levels in healthy controls, patients with slow or rapid progressing disease and patients with dementia; (B) serum sCD14 levels in healthy controls, patients with mild Alzheimer's disease (Mild AD; n=10), patients with advanced Alzheimer's disease (Adv AD), and patients with frontal temporal dementia (FTD); (C) serum sCD14 levels in healthy controls and chronic inflammatory demyelinating polyneuropathy (CIDP) patients; and (D) serum sCD14 levels in healthy controls, ALS patients with slow or rapid progressing disease, and CIDP patients.

FIG. 7 shows the correlations between serum sCD14 and markers of disease burden or disease progression in ALS patients. (A) Correlation between serum sCD14 and AALS score in ALS patients; (B) Correlation between serum sCD14 and suppressive capabilities of ALS Tregs; and (C) ROC curve for predicting slow versus rapid progression of disease using serum sCD14 levels.

FIG. 8 shows the correlation between serum sCD14 levels and clinical outcome in subjects with ALS, using an ROC score cutoff of 2.73 μg/ml of serum sCD14. (A) Percentage of ALS patients with sCD14 levels above 2.73 μg/ml that were deceased or alive; (B) Time from diagnosis to an AALS score of 100 points for ALS patients with sCD14 levels above or below 2.73 μg/ml; (C) Time from diagnosis to death for ALS patients with sCD14 levels above or below 2.73 μg/ml; (D) Correlation between serum sCD14 and time to reach an AALS score of 100 points from diagnosis in individual ALS patients; (E) Correlation between serum sCD14 and time from diagnosis to death in individual ALS patients.

FIG. 9 shows the level of lipopolysaccharide binding protein (LBP) in sera of healthy volunteers (controls) and ALS subjects and correlation with disease burden. (A) Serum levels of LBP in controls and ALS patients; (B) Serum levels of LBP in controls and ALS patients with slow or rapid progressing disease. (C) Correlation between serum levels of LBP and AALS score; and (D) Correlation between serum levels of LBP and serum levels of sCD14.

FIG. 10 shows the level of C-reactive protein (CRP) in sera of healthy volunteers (healthy controls) and ALS subjects. (A) Serum levels of CRP in healthy controls and ALS patients; (B) Serum levels of CRP in healthy controls and ALS patients with slow or rapid progressing disease.

FIG. 11 shows the level of macrophage migration inhibitory factor (MIF) in sera of healthy volunteers (healthy controls) and ALS subjects. (A) Serum levels of MIF in healthy controls and ALS patients; (B) Serum levels of MIF in healthy controls and ALS patients with slow or rapid progressing disease.

FIG. 12 shows the level of soluble tumor necrosis factor receptor I and II (sTNFRI and sTNFRII) in sera of healthy volunteers (healthy controls) and ALS subjects. (A) Serum levels of sTNFRI in healthy controls and ALS patients; (B) Serum levels of sTNFRII in HV and ALS patients; (C) Serum levels of TNFRI in healthy controls and ALS patients with slow or rapid progressing disease; (D) Serum levels of TNFRII in healthy controls and ALS patients with slow or rapid progressing disease; and (E) Correlation between serum levels of TNFRI and TNFRII in ALS patients.

FIG. 13 provides levels of sCD14 in serum samples from healthy volunteers (healthy controls or control (C)) and subjects with ALS (second study). (A) Serum sCD14 levels in patients with ALS or healthy controls from the first or second study. (B) Serum sCD14 levels in patients with slow or fast progressing ALS or healthy controls, comparing cohorts from the first and the second study. (C) Correlation between serum sCD14 and rate of progression of disease in ALS patients in the second study. (D) ROC curve for predicting rapid (fast) or slow progression versus control using serum sCD14 levels (P<0.0001 by Mann Whitney test; Sensitivity: 0.767; Specificity: 0.710). (E) ROC curve for predicting rapid (fast) versus slow progression using serum sCD14 levels (P<0.0001 by Mann Whitney test; Sensitivity: 0.942; Specificity: 0.958). (F) ROC curve for predicting rapid (fast) progression versus control using serum sCD14 levels (P<0.0001 by Mann Whitney test; Sensitivity: 0.950; Specificity: 0.958). (G) ROC curve for predicting slow progression versus control using serum sCD14 levels (P<0.0001 by Mann Whitney test; Sensitivity: 0.633; Specificity: 0.654).

FIG. 14 provides levels of LBP in serum samples from healthy volunteers (healthy controls or control (C)) and subjects with ALS (second study). (A) Serum LBP levels in patients with ALS or healthy controls from the first or second study. (B) Serum LBP levels in patients with slow or fast progressing ALS or healthy controls, comparing cohorts from the first and the second study. (C) Correlation between serum LBP and rate of progression of disease in ALS patients in the second study. (D) ROC curve for predicting rapid (fast) or slow progression versus control using serum LBP levels (P<0.0001 by Mann Whitney test; Sensitivity: 0.817; Specificity: 0.920). (E) ROC curve for predicting rapid (fast) versus slow progression using serum LBP levels (P<0.0001 by Mann Whitney test; Sensitivity: 0.923; Specificity: 0.938). (F) ROC curve for predicting rapid (fast) progression versus control using serum LBP levels (P<0.0001 by Mann Whitney test; Sensitivity: 0.967; Specificity: 1). (G) ROC curve for predicting slow progression versus control using serum LBP levels (P<0.0001 by Mann Whitney test; Sensitivity: 0.783; Specificity: 0.865).

FIG. 15 shows the correlation between sCD14 and LBP in the serum of slow and fast progressing ALS patients.

FIG. 16 shows ROC curves for predicting progression of disease using the combination of serum sCD14 and serum LBP. (A) ROC curve for predicting rapid (fast) or slow (S) progression versus control using serum sCD14 and LBP levels. (B) ROC curve for predicting rapid (fast; F) versus slow (S) progression using serum sCD14 and LBP levels. (C) ROC curve for predicting rapid (fast; F) progression versus control (C) using serum sCD14 and LBP levels. (D) ROC curve for predicting slow (S) progression versus control (C) using serum sCD14 and LBP levels. (E) ROC curve for predicting rapid (fast; F) or slow (S) progression versus control (C) using scaled serum sCD14 and scaled serum LBP levels (P<0.0001 by Mann Whitney test; Sensitivity: 0.783; Specificity: 0.950). (F) ROC curve for predicting rapid (fast; F) versus slow (S) progression using scaled serum sCD14 and scaled serum LBP levels (P<0.0001 by Mann Whitney test; Sensitivity: 0.942; Specificity: 0.958). (G) ROC curve for predicting rapid (fast; F) progression versus control (C) using scaled serum sCD14 and scaled serum LBP levels (P<0.0001 by Mann Whitney test; Sensitivity: 1; Specificity: 1). (H) ROC curve for predicting slow (S) progression versus control (C) using scaled serum sCD14 and scaled serum LBP levels (P<0.0001 by Mann Whitney test; Sensitivity: 0.800; Specificity: 0.808).

### DETAILED DESCRIPTION OF THE DISCLOSURE

### 1. Definitions

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the present disclosure, preferred methods and materials are described. For the purposes of the present disclosure, the following terms are defined below.

The articles “a” and “an” are used herein to refer to one or to more than one (i.e. to at least one) of the grammatical object of the article. By way of example, “a biomarker” means one biomarker or more than one biomarker.

As used herein, “and/or” refers to and encompasses any and all possible combinations of one or more of the associated listed items, as well as the lack of combinations when interpreted in the alternative (or).

The terms “ALS” and “Lou Gehrig's disease” may be used interchangeably herein to refer to the same condition. Both familial ALS and sporadic ALS may be treated or its development or progression determined by the subject method. All forms of ALS are contemplated herein. Subjects with ALS can have rapid progressing ALS (a term used interchangeably herein with fast progressing ALS), or slow progressing ALS, terms which are well known in the art. Those skilled in the art would appreciate that the precise categorisation of slow and rapid progressing subjects will depend on the scoring system used to assess the disease and disease progression. Exemplary scoring systems include, for example, the examination-based Appel ALS (AALS) score and the questionnaire-based ALS Functional Rating Scale (ALSFRS) score. The ALSFRS score is based on ten questions grouped into four areas (fine motor, gross motor, bulbar, and respiratory) and ranges from 40 (normal) to 0 (lowest functioning) (Cedarbaum et al. J Neurol Sci 1997; 152(suppl 1):S1-9). The AALS score is based on objective testing in five categories (bulbar, respiratory function, arm and leg function, and muscle strength) and ranges from 30 (normal) to 164 (maximally impaired) (Appel et al. Ann Neurol 1987; 22:328-333; Haverkamp et al. Brain. 1995; 118:707-19). In particular examples, the AALS scoring system is used, and a threshold value distinguishes rapid progressing ALS from slow progressing ALS. The threshold value may be, for example, from about 1.0 to about 2.0 AALS points/month, such as 1.0, 1.1, 1.2, 1.3, 1.4, 1.5, 1.6, 1.7, 1.8, 1.9 or 2.0 AALS points/month, wherein a subject with rapid progressing ALS is one progressing at a rate of greater than or equal to the threshold, and a subject with slow progressing ALS is one progressing at a rate of less than the threshold. In other examples, a subject with rapid progressing ALS is one progressing at a rate of greater than the threshold, and a subject with slow progressing ALS is one progressing at a rate of less than or equal to the threshold. In a particular embodiment, the threshold value for distinguishing rapid progressing ALS from slow progressing ALS is 1.5 AALS points/month, where a subject with rapid progressing ALS is one progressing at a rate of greater than or equal to 1.5 AALS points/month, and a subject with slow progressing ALS is one progressing at a rate of less than 1.5 AALS points/month (Henkel et al., EMBO Mol Med 2013; 5:64-79).

The “amount” or “level” of a biomarker is a detectable level in a sample and may represent an absolute amount or level or a relative amount or level. These can be measured by methods known to one skilled in the art, illustrative examples of which are disclosed herein.

As used herein, “and/or” refers to and encompasses any and all possible combinations of one or more of the associated listed items, as well as the lack of combinations when interpreted in the alternative (or).

By “antigen-binding molecule” is meant a molecule that has binding affinity for a target antigen. It will be understood that this term extends to immunoglobulins, immunoglobulin fragments and non-immunoglobulin derived protein frameworks that exhibit antigen-binding activity. Representative antigen-binding molecules that are useful in the practice of the present invention include antibodies and antigen-binding fragments thereof.

The term “antibody” herein is used in the broadest sense and specifically covers monoclonal antibodies (including full length monoclonal antibodies), polyclonal antibodies, chimeric antibodies, humanized antibodies, human antibodies, multispecific antibodies (e.g., bispecific antibodies), and single variable domain antibodies so long as they exhibit the desired biological activity. The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (which may be abbreviated as HCVR or VH) and a heavy chain constant region. The heavy chain constant region comprises three domains, C_{H1}, C_{H2} and C_{H3}. Each light chain comprises a light chain variable region (which may be abbreviated as LCVR or V_{L}) and a light chain constant region. The light chain constant region comprises one domain (C_{L1}). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the invention, the FRs of an antibody (or antigen-binding portion thereof) may be identical to the human germline sequences, or may be naturally or artificially modified. An amino acid consensus sequence may be defined based on a side-by-side analysis of two or more CDRs. Included within the scope of the term “antibody” is an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called α, δ, ε, γ, and μ, respectively. The subunit structures and three-dimensional configurations of different classes of immunoglobulins are well known.

An “antigen-binding fragment” may be provided by means of arrangement of one or more CDRs on non-antibody protein scaffolds. “Protein Scaffold” as used herein includes but is not limited to an immunoglobulin (Ig) scaffold, for example an IgG scaffold, which may be a four chain or two chain antibody, or which may comprise only the Fc region of an antibody, or which may comprise one or more constant regions from an antibody, which constant regions may be of human or primate origin, or which may be an artificial chimera of human and primate constant regions. The protein scaffold may be an Ig scaffold, for example an IgG, or IgA scaffold. The IgG scaffold may comprise some or all the domains of an antibody (i.e. CH1, CH2, CH3, V_{H}, V_{L}). The antigen binding protein may comprise an IgG scaffold selected from IgG1, IgG2, IgG3, IgG4 or IgG4PE. For example, the scaffold may be IgG1. The scaffold may consist of, or comprise, the Fc region of an antibody, or is a part thereof. Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment,” as used herein. An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain. In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H1}; (ii) V_{H}-C_{H2}; (iii) V_{H}-C_{H3}; (iv) V_{H}-C_{H1}-C_{H2}; (V) V_{H}-C_{H1}-C_{H2}-C_{H3}; (Vi) V_{H}-C_{H2}-C_{H3}; (Vii) V_{H}-C_{L}; (Viii) V_{L}-C_{H1}; (iX) V_{L}-C_{H2}; (X) V_{L}-C_{H3}; (Xi) V_{L}-C_{H1}-C_{H2}; (XII) V_{L}-C_{H1}-C_{H2}-C_{H3}; (Xiii) V_{L}-C_{H2}-C_{H3}; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)). As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antigen-binding molecule format, including the exemplary bispecific antigen-binding molecule formats disclosed herein, may be adapted for use in the context of an antigen-binding fragment of an antibody of the present invention using routine techniques available in the art.

The terms “binds to”, “specifically binds to,” “specific for,” and related grammatical variants typically refer to that binding which occurs between such paired species (e.g., antibody and antigen) which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs.

As used herein, the term “biomarker” refers to a molecule that is associated, either quantitatively or qualitatively, with a particular disease, phenotype, biological activity or function (e.g., the presence of ALS, symptoms thereof, the severity of ALS, and the rate of disease progression). Illustrative examples of suitable biomarkers include proteins, polypeptides, and fragments of a polypeptide or protein; carbohydrates, and/or glycolipid-based molecular markers; polynucleotides, such as a gene product, RNA or RNA fragment, polynucleotide copy number alterations (e.g., DNA copy numbers); polynucleotide or polypeptide modifications (e.g., posttranslational modifications, phosphorylation, DNA methylation, acetylation, and other chromatin modifications, glycosylation, etc.). In certain embodiments, a “biomarker” means a molecule/compound that is differentially present (i.e., increased/upregulated or decreased/downregulated) in a sample as measured/compared against the same marker in another sample or a suitable control/reference. In other embodiments, a biomarker can be differentially present in a sample as measured/compared against the other markers in same or another sample or suitable control/reference. In further embodiments, biomarkers can be differentially present in a sample as measured/compared against other markers in the same or another sample or suitable control/reference and against the same markers in another sample or suitable control/reference. In yet another embodiment, a biomarker can be differentially present in a sample from a subject or a group of subjects having a first phenotype (e.g., having a disease or condition) as compared to a sample from a subject or group of subjects having a second phenotype (e.g., not having the disease or condition or having a less severe version of the disease or condition). An “ALS progression biomarker” refers to a molecule that is associated with the rate of ALS progression.

Throughout this specification, unless the context requires otherwise, the words “comprise,” “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

By “effective amount”, in the context of treating a condition is meant the administration of an amount of an agent or composition to an individual in need of such treatment or prophylaxis, either in a single dose or as part of a series, that is effective for the prevention of incurring a symptom, holding in check such symptoms, and/or treating existing symptoms, of that condition. The effective amount will vary depending upon the age, health and physical condition of the individual to be treated and whether symptoms of disease are apparent, the taxonomic group of individual to be treated, the formulation of the composition, the assessment of the medical situation, and other relevant factors. Optimal dosing schedules can be calculated from measurements of drug accumulation in the body of the subject. Optimum dosages may vary depending on the relative potency in an individual subject, and can generally be estimated based on EC50 values found to be effective in in vitro and in vivo animal models. Persons of ordinary skill can easily determine optimum dosages, dosing methodologies and repetition rates. It is expected that the amount will fall in a relatively broad range that can be determined through routine trials.

The terms “elevated level” and the like refers to an increased level or amount of a biomarker in a sample relative to a suitable reference level, such as the level of the same biomarker in a sample from a subject known to have ALS at the time of sampling or from a healthy subject know to be free of ALS.

The term “reduced level” and the like refers to a decreased level or amount of a biomarker in a sample relative to a suitable reference level, such as the level of the same biomarker in a sample derived from a subject know to have ALS at the time of sampling or from a healthy subject know to be free of ALS.

As used herein, reference to levels of a biomarker in a sample that are “the same or similar” relative to a suitable reference level means that any difference in the level of the biomarker in the sample and the reference level is insufficient to distinguish them from one another, or to distinguish the condition or phenotype that they represent (e.g. healthy, slow progressing ALS, or rapid progressing ALS). As would be appreciated, this can be determined using mathematical models and/or statistical analysis. In a particular example, the “same or similar” means no more than 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 50%, 60%, 70%, 80%, 90%, 100% or more difference in levels.

As used herein, “instructional material” includes a publication, a recording, a diagram, or any other medium of expression which can be used to communicate the usefulness of the compositions and methods of the disclosure. The instructional material of the kit of the disclosure may, for example, be affixed to a container which contains the nucleic acid, peptide, and/or composition of the disclosure or be shipped together with a container which contains the nucleic acid, peptide, and/or composition. Alternatively, the instructional material may be shipped separately from the container with the intention that the instructional material and the compound be used cooperatively by the recipient.

The term “monocyte”, as used herein, refers to a type of white blood cell of the immune system. Monocytes are typically characterised by the expression of CD14. Monocytes may also express of one or more of the following cell surface markers: 125I-WVH-1, 63D3, Adipophilin, CB12, CD11a, CD11b, CD15, CD54, Cd163, cytidine deaminase, Flt-1, and the like. The term “monocyte” includes, without limitation, both the classical monocyte and the non-classical pro-inflammatory monocyte, which are present in human blood. By “classical monocyte” is meant a type of monocyte characterized by a high level of cell surface CD14 expression (CD14++ monocyte), whereas as the term “non-classical pro-inflammatory monocyte” typically means a monocyte with a low level cell surface CD14 expression, optionally with additional co-expression of the cell surface CD16 receptor (CD14+CD16+ monocyte).

The terms “patient” and “subject” are used interchangeably herein and refer broadly to any vertebrate animal. Suitable vertebrate animals will be familiar to persons skilled in the art, illustrative examples of which include a member of the subphylum Chordata including primates (e.g., humans, monkeys and apes). In a preferred embodiment, the subject is a human. In some embodiments, the subject has ALS, such as rapid progressing ALS or slow proressing ALS.

The terms “polynucleotide,” “genetic material,” “genetic forms,” “nucleic acids” and “nucleotide sequence” include RNA, cDNA, genomic DNA, synthetic forms and mixed polymers, both sense and antisense strands, and may be chemically or biochemically modified or may contain non-natural or derivatized nucleotide bases, as will be readily appreciated by those skilled in the art.

The term “prognostic” and grammatical variations thereof generally refer to a biomarker(s), biomarker profile or method that provides information regarding the likely progression or severity of a disease or condition, such as ALS, in an individual. In some embodiments, prognosis also refers to the ability to demonstrate a positive or negative response to therapy or other treatment regimens, for the disease or condition in the subject. In some embodiments, prognosis refers to the ability to predict the presence or diminishment of disease/condition associated symptoms. A prognostic agent or method may comprise classifying a subject or sample obtained from a subject into one of multiple categories, wherein the categories correlate with different likelihoods that a subject will experience a particular outcome. For example, categories can be low risk and high risk, wherein subjects in the low risk category have a lower likelihood of experiencing a poor outcome (e.g., within a given time period such as 5 years or 10 years) than do subjects in the high risk category. A poor outcome could be, for example, disease progression, disease recurrence, or death attributable to the disease. By “likelihood” is meant a measure of whether a subject with particular biomarker level actually has rapid or slow progressing ALS based on a given mathematical model. An increased likelihood for example may be relative or absolute and may be expressed qualitatively or quantitatively. For instance, an increased likelihood may be determined simply by determining the level of a biomarker and placing the subject in an “increased likelihood” category, based upon previous population studies. The term “likelihood” is also used interchangeably herein with the term “probability”.

As used herein, the rate of progression of ALS refers is an assessment of the time taken for the severity of the disease to increase. The severity of disease can be assessed by measuring the number of symptoms of disease that a patient presents with, and/or the severity of any one or more symptoms. Various scoring systems can be used to assess the severity of disease, including the examination-based Appel ALS (AALS) score and the questionnaire-based ALS Functional Rating Scale (ALSFRS) score (discussed above). The rate of progression in such circumstances can be determined by calculating the change in score over time, e.g. change in AALS score per month. As described herein, biomarkers such as sCD14 and/or LBP strongly correlate with the rate of disease progression and levels of such biomarkers can therefore also be used to assess the rate of progression of ALS.

The term “sample”, as used herein, includes any biological specimen comprising a biological marker as herein described that may be extracted, untreated, treated, diluted or concentrated from a subject. The sample may contain one biological marker (e.g. sCD14) or a population of biological markers. Illustrative examples of biological samples contemplated for use in accordance with the present disclosure include blood, serum, plasma, urine, cerebrospinal fluid (CSF) and saliva.

The methods disclosed herein may include a step that involves comparing a value or level of a biomarker in a sample to a “reference level”, referred to interchangeably as a “reference”, “suitable control”, “control” etc. A “reference level” is a value, level or range of values or levels that is/are representative of a phenotype or condition, such as a healthy condition, rapid progressing ALS or slow progressing ALS. In some embodiments, the reference value is determined from one or more biological samples taken from one or more subjects with rapid progressing ALS, one or more subjects with slow progressing ALS, or one or more subjects without ALS, such as one or more healthy individuals. In other embodiments, a “reference value” is a pre-determined or pre-defined value or level that has been established as being reflective of, for example, a healthy subject, a subject with rapid progressing ALS or a subject with slow progressing ALS. (e.g., biomarker levels that correlate to a particular biomarker profile). Thus, in some embodiments, the reference level has been determined prior to performing the methods disclosed herein. Reference levels or values may also be tailored to specific techniques that are used to measure levels of biomarkers in biological samples (e.g., ELISA, LC-MS, GC-MS, PCR, etc.), where the levels of biomarkers may differ based on the specific technique that is used. In a particular embodiment, a reference level is a threshold level or value, above which or below which is indicative of rapid progressing ALS or slow progressing ALS.

The term “solid support” as used herein refers to a solid inert surface or body to which a molecular species, such as a nucleic acid and/or polypeptide can be immobilized. Non-limiting examples of solid supports include glass surfaces, plastic surfaces, latex, dextran, polystyrene surfaces, polypropylene surfaces, polyacrylamide gels, gold surfaces, and silicon wafers. In some embodiments, the solid supports are in the form of membranes, slides, chips, particles (including beads), and multiwall plates. A support can include a plurality of particles or beads, each having a different attached molecular species. In some embodiments, the solid support may comprise an inert substrate or matrix which has been “functionalized”, such as by applying a layer or coating of an intermediate material comprising reactive groups which permit covalent attachment to molecules. The molecules can be directly covalently attached to the intermediate material but the intermediate material can itself be non-covalently attached to the substrate or matrix.

As used herein, reference to a “symptom” of ALS disease is a physical or mental feature which is regarded as indicating ALS disease. Non-limiting ALS-mediated disease symptoms include progressive muscle atrophy, paralysis, spasticity, hyperreflexia, and other symptoms such as difficulty swallowing, limb weakness, slurred speech, impaired gait, facial weakness, respiratory changes and muscle cramps. Typically, a subject will present with one or several symptoms depending on the disease severity, clinical stage of disease and the individual subject. Determination of which symptoms are regarded as indicating ALS disease would be well within the skill of those in the art.

As used herein, the terms “treatment”, “treating”, and the like, refer to obtaining a desired pharmacologic and/or physiologic effect in a subject in need of treatment, that is, a subject who has ALS or is diagnosed as having ALS or a subject at risk of developing ALS. By “treatment” is meant:
(a) delaying development and/or progression of ALS;
(b) ameliorating symptoms of ALS;
(c) suppressing ALS or its symptoms; and/or
(d) improving or prolonging quality of life.

Reference to “treatment”, “treat” or “treating” does not necessarily mean to cure the subject or prevent disease progression indefinitely. The subject may ultimately succumb to the neurodegenerative disease, however, the quality of life is extended for a period longer than without treatment since the development of the disease or condition is delayed.

Indicia of successful “treatment”, includes any objective or subjective parameter such as abatement; remission; diminishing of memory or condition more tolerable to the patient; slowing in the rate of degeneration or decline or worsening of the illness; making the final point of worsening less debilitating; or improving a subject's physical or mental well-being. The treatment or amelioration of symptoms can be based on objective or subjective parameters; including the results of a physical examination, neurological examination, and/or psychiatric evaluations.

Each embodiment described herein is to be applied mutatis mutandis to each and every embodiment unless specifically stated otherwise.

### 2. Methods for Assessing the Rate of Progression of ALS

The present disclosure is predicated, at least in part, on the determination that the level of one or more biomarkers (i.e. ALS progression biomarkers) in a biological sample of a subject with ALS correlates with the rate of progression of ALS in the subject. For example, as demonstrated herein, the level of one or more biomarkers in a biological sample of a subject with rapid progressing ALS is typically increased (or elevated) compared to the level of the same biomarker in a biological sample of a subject with slow progressing ALS and compared to a healthy subject. Conversely, the level of one or more biomarkers in a biological sample of a subject with slow progressing ALS is typically decreased (or reduced) compared to the level of the same biomarker in a biological sample of a subject with rapid progressing ALS. Depending upon the biological sample being assessed, and as taught herein, the level of one or more biomarkers of a subject with slow progressing ALS may be the same or similar, or increased, compared to the level of the same biomarker in a biological sample of a healthy subject. Accordingly, the methods of the present disclosure include methods for assessing the rate of progression of ALS in a subject by determining the level of one or more ALS progression biomarkers in a biological sample and determining the rate of progression based on the level of the one or more ALS progression biomarkers in the biological sample, such as relative to a reference level (where the reference level is indicative of or represents a particular rate of progression). The methods of the present disclosure also include methods for determining the likelihood of a subject having rapid or slow progressing ALS by determining the level of one or more ALS progression biomarkers in a biological sample and determining whether or not the subject is likely to have rapid or slow progressing ALS based on the level of the one or more ALS progression biomarkers in the biological sample relative to a reference level. Typically, the subject has already been diagnosed with ALS or is simultaneously being diagnosed with ALS, e.g., the assessment of the ALS progression biomarkers as described herein can be made in conjunction with other assessments to confirm a diagnosis of ALS. In further embodiments, the subject is undergoing treatment for ALS, and the assessment of the rate of progression of ALS is used to assess the efficacy of treatment.

In some embodiments of any of the methods described herein, an increased or elevated level refers to an overall increase of at least or about any of 10%, 20%, 30%, 40%, 50%, 60%, 70%, 80%, 90%, 95%, 96%, 97%, 98%, 99% or greater, in the level of biomarker, detected by standard art-known methods such as those described herein, as compared to a reference level. In certain embodiments, an increased or elevated level refers to the increase in the level/amount of a biomarker in the sample wherein the increase is at least or about any of 1.5×, 1.75×, 2×, 3×, 4×, 5×, 6×, 7×, 8×, 9×, 10×, 25×, 50×, 75×, or 100× of a reference level.

In some embodiments of any of the methods described herein, a decreased or reduced level refers to an overall reduction of at least or about any of 10%, 20%, 30%, 40%, 50%, 60%, 70%, 80%, 90%, 95%, 96%, 97%, 98%, 99% or greater, in the level of biomarker, detected by standard art-known methods such as those described herein, as compared to a reference level. In certain embodiments, a decreased or reduced level refers to a decrease in level/amount of a biomarker in the sample wherein the decrease is at least or about any of 0.9×, 0.8×, 0.7×, 0.6×, 0.5×, 0.4×, 0.3×, 0.2×, 0.1×, 0.05×, or 0.01× of a reference level.

### Biomarkers

Exemplary of the ALS progression biomarkers that are prognostic of the rate of progression of ALS is soluble CD14 (sCD14). CD14, a glycosylphosphatidylinositol (GPI)-anchored membrane glycoprotein, is a myeloid differentiation marker expressed predominately by monocytes/macrophages, although low levels are also found on neutrophils. Along with the Toll-like receptor 4 and MD-2, CD14 acts as a co-receptor for LPS. Upon monocyte activation, membrane CD14 (mCD14) is cleaved from the cell surface and sCD14 is released. Monocyte activation-dependent shedding of mCD14 yields the vast majority of sCD14.

As demonstrated herein, the level of sCD14 in a biological sample from a subject with ALS can be used to assess the rate of progression of ALS and can be used to determine the likelihood of that subject having rapid or slow progressing ALS. Levels of sCD14 strongly correlate with disease progression, with lower levels of sCD14 correlating with slower progression, and higher levels of sCD14 correlating with faster progression. Thus, subjects that have rapid progressing ALS typically have an increased (or elevated) level of sCD14 compared to subjects with slow progressing ALS and healthy subjects. Subjects that have slow progressing ALS typically have a decreased (or lowered) level of sCD14 compared to subjects with rapid progressing ALS. The biological sample may be, for instance, blood, serum, plasma, urine or CSF.

In instances where the biological sample is blood, serum, plasma or CSF, subjects that have slow progressing ALS may have the same or similar level of sCD14 compared to healthy subjects, or may have increased levels of sCD14 compared to healthy subjects. Where the biological sample is urine, subjects that have slow progressing ALS typically have an increased (or elevated) level of sCD14 compared to healthy subjects, albeit not so increased as the levels observed in subjects that have rapid progressing ALS (i.e. the levels of sCD14 in the urine of subjects with slow progressing ALS is typically increased compared to healthy individuals but decreased compared to subjects with rapid progressing ALS).

Thus, a determination of the level of sCD14 in a biological sample from a subject with ALS can be used to assess the rate of progression of ALS in a subject, and/or to determine the likelihood of that subject having rapid progressing ALS or slow progressing ALS.

Lipopolysaccharide binding protein (LBP, also referred to herein as sLBP) is another ALS progression biomarker that has prognostic value in relation to progression rates of ALS. Lipopolysaccharide binding protein is a soluble acute-phase protein that binds LPS. CD14 is a co-receptor for LPS, but can only bind LPS in the presence of LBP. LBP is synthesized by hepatocytes and intestinal epithelial cells, and for signal transduction, binding complexes of LPS and LBP to CD14 is necessary.

Levels of LBP also strongly correlate with disease progression, with lower levels of LBP correlating with slower progression, and higher levels of LBP correlating with faster progression. Generally, LBP levels are typically increased (or elevated) in subjects that have rapid progressing ALS as compared to either subjects with slow progressing ALS or healthy subjects. Subjects that have slow progressing ALS typically have decreased (or reduced) levels of LBP compared to subjects with rapid progressing ALS, and the same or similar levels of LBP as healthy subjects or increased levels of LBP compared to healthy subjects. Thus, a determination of the level of LBP in a biological sample from a subject with ALS can be used to assess the rate of progression of ALS in a subject and/or to determine the likelihood of that subject having rapid progressing ALS or slow progressing ALS.

A further exemplary biomarker that has prognostic value for ALS progression is C reactive protein (CRP). CRP is produced by the liver and increases in the presence of inflammation. Subjects that have rapid progressing ALS typically have an increased (or elevated) level of CRP compared to subjects with slow progressing ALS and healthy subjects. Subjects that have slow progressing ALS typically have decreased (or reduced) levels of CRP in a biological sample compared to subjects with rapid progressing ALS, and the same or similar levels of CRP as healthy subjects. Thus, a determination of the level of CRP in a biological sample from a subject with ALS can be used to determine the likelihood of that subject having rapid progressing ALS or slow progressing ALS.

Soluble tumor necrosis factor receptor I (sTNFR1) and soluble tumor necrosis factor receptor II (sTNFRII) may also be used in accordance with the present disclosure as prognostic biomarkers for ALS progression. TNFRI and TNFRII are cell surface receptors that bind TNF. Both receptors are ubiquitously expressed and display structurally similar extracellular domains but signal through distinct intracellular regions, with TNFRI containing a death domain that is not present in TNFRII. The two TNFRs are released as soluble proteins by proteolytic cleavage of their extracellular domains, in exosomes, or via alternative splicing of mRNA transcripts which leads to a loss of the transmembrane and cytoplasmic domains.

As demonstrated herein, levels of sTNFRI and sTNFRII are typically increased (or elevated) in subjects that have rapid progressing ALS as compared to either subjects with slow progressing ALS or healthy subjects. Subjects that have slow progressing ALS typically have decreased (or reduced) levels of sTNFRI and sTNFRII compared to subjects with rapid progressing ALS, and the same or similar levels as healthy subjects. Thus, a determination of the level of TNFRI and/or TNFRII in a biological sample from a subject with ALS can be used to determine the likelihood of that subject having rapid progressing ALS or slow progressing ALS.

In particular embodiments, the methods of the present disclosure comprise determining the level of at least sCD14 in a biological sample. In other embodiments, the methods of the present disclosure comprise determining the level of at least LBP in a biological sample. In a particular embodiment, the levels of both sCD14 and LBP are measured. Optionally, such methods can further include determining the level of at least one other biomarker, such as one or more of CRP, TNFRI and TNFRII. In alternative embodiments, the methods of the present disclosure comprise determining the level of at least TNFRI or TNFRII, optionally with one or more of sCD14, LBP and CRP.

Other biomarkers that may be assessed in the methods of the present disclosure include, for example, the pro-inflammatory cytokine MIF (which, as demonstrated herein, is typically elevated in both subjects with rapid progressing ALS and subjects with slow progressing ALS as compared to healthy subjects), neurofilament light chain (NFL; Tortelli et al., Eur J Neurol. 2012; 19(12):1561-7; Gaiani et al. JAMA Neurol. 2017, 74(5):525-532), miR-206, miR-143-3p, and/or miR-374b-5p (Waller et al. Neurobiol Aging 2017, 55:123-131), phosphorylated neurofilament heavy chain (pNfH) (Boylan et al., J Neurochem. 2009; 111(5):1182-91; Steinacker et al. J Neurol Neurosurg Psychiatry. 2016, 87(1):12-20), and/or p75 neurotrophin receptor extracellular domain (p75NTR^{ECD}; Shepheard et al. Neurology. 2017, 88(12):1137-1143). These additional biomarkers may provide further clarity on the disease burden and/or rate of progression, as described herein and previously.

### Biological Samples

A biological sample may include a sample that is extracted, untreated, treated, diluted or concentrated from a subject. Suitable biological samples for assessment of the one or more biomarkers of the present disclosure include, but are not limited, blood, serum, plasma, urine and CSF. In particular embodiments, the level of the one or more ALS progression biomarkers in serum is assessed. In other exemplary embodiments, the level of the one or more ALS progression biomarkers in urine is assessed. Methods for obtaining biological samples are well-known in the art.

It is understood that in instances where two or more biomarkers are assessed, the two or more biomarkers may be assessed in the same biological sample or in different biological samples from the same subject. This includes biological samples of the same type taken at different times (e.g. two serum samples taken at different times, although typically within minutes or hours of one another), and biological samples of different types (e.g. a serum sample and a urine sample).

### Assessment of Biomarker Levels

Determining the level of one or more biomarkers in accordance with the present disclosure encompasses obtaining, such as from a database, computer, or communication or report from a technician or laboratory, a previously-measured level of one or more biomarkers. Determining the level of one or more biomarkers in accordance with the present disclosure also encompasses measuring the level of one or more biomarkers. Thus, in particular embodiments of the present disclose, the level of one or more biomarkers in a biological sample is measured so as to determine the level of the one or more biomarkers.

The level of the one or more biomarkers may be measured or assessed using any appropriate technique or means known to those of skill in the art. In particular embodiments, the level of a biomarker, such as sCD14, LBP, CRP, MIF, sTNFRI and/or sTNFRII, is assessed using an antibody-based technique, non-limiting examples of which include immunoassays, such as the enzyme-linked immunosorbent assay (ELISA) and the radioimmunoassay (RIA). A wide range of immunoassay techniques using such an assay format are available, see, e.g., U.S. Pat. Nos. 4,016,043, 4,424,279 and 4,018,653. These include both single-site and two-site or “sandwich” assays of the non-competitive types, as well as in the traditional competitive binding assays. These assays also include direct binding of a labelled antibody to a target biomarker. ELISAs for measuring the levels of sCD14, LBP, CRP, MIF, sTNFRI and/or sTNFRII are available commercially and/or can be readily developed by those skilled in the art using known antibodies specific for sCD14, LBP, CRP, MIF, sTNFRI and/or sTNFRII

In specific embodiments, where the levels of two or more biomarkers are assessed, a multiplex assay, such as a multiplex immunoassay (e.g. multiplex ELISA), can be employed. Multiplex assays include arrays comprising spatially addressed antigen-binding molecules, commonly referred to as antibody arrays, which can facilitate extensive parallel analysis of multiple proteins. Antibody arrays have been shown to have the required properties of specificity and acceptable background. Various methods for the preparation of antibody arrays have been reported (see, e.g., Lopez et al., 2003 J. Chromatogr. B 787:19-27; Cahill, 2000 Trends in Biotechnology 7:47-51; U.S. Pat. App. Pub. 2002/0055186; U.S. Pat. App. Pub. 2003/0003599; PCT publication WO 03/062444; PCT publication WO 03/077851; PCT publication WO 02/59601; PCT publication WO 02/39120; PCT publication WO 01/79849; PCT publication WO 99/39210).

Individual spatially distinct protein-capture agents are typically attached to a support surface, which is generally planar or contoured. Common physical supports include glass slides, silicon, microwells, nitrocellulose or PVDF membranes, and magnetic and other microbeads.

Particles in suspension can also be used as the basis of multiplex assays and arrays, providing they are coded for identification; systems include colour coding for microbeads (e.g., available from Luminex, Bio-Rad and Nanomics Biosystems) and semiconductor nanocrystals (e.g., QDots™, available from Quantum Dots), and barcoding for beads (UltraPlex™, available from Smartbeads) and multimetal microrods (Nanobarcodes™ particles, available from Surromed). Beads can also be assembled into planar arrays on semiconductor chips (e.g., available from LEAPS technology and BioArray Solutions). Where particles are used, individual protein-capture agents are typically attached to an individual particle to provide the spatial definition or separation of the array. The particles may then be assayed separately, but in parallel, in a compartmentalized way, for example in the wells of a microtitre plate or in separate test tubes.

One illustrative example of a protein-capture array is Luminex®-based multiplex assay, which is a bead-based multiplexing assay, where beads are internally dyed with fluorescent dyes to produce a specific spectral address. Biomolecules (such as an antibody) can be conjugated to the surface of beads to capture biomarkers of interest. Flow cytometric or other suitable imaging technologies known to persons skilled in the art can then be used for characterization of the beads and detection and quantitation of the biomarkers.

Other methods for detecting and quantitating protein biomarkers include, but are not limited to, mass spectrometry (MS) methods, including Liquid Chromatography-Mass Spectrometry (LC-MS), Direct Analysis in Real Time Mass Spectrometry (DART MS), SELDI-TOF and MALDI-TOF.

Assessment of progression of ALS

The methods of the present disclosure can be used to assess the likely rate of progression of ALS in a subject, and can be used to determine whether a subject with ALS is likely to have slow or rapid progressing ALS. This is done by determining the level of one or more ALS progression biomarkers as described above and herein and optionally comparing that level to a suitable reference level. As would be appreciated, whether an increased (or elevated), decreased (or reduced) or the same or similar level of a biomarker compared to a reference level indicates that the subject is likely to have slow progressing ALS or rapid progressing ALS, or indicates what rate of progression the subject has, will depend on the reference level being used in the assessment.

Reference levels may be representative of a healthy subject, a subject with slow progressing ALS, a subject with rapid progressing ALS, or a subject with a particular progression rate (e.g. as measured by AALS points per month). Thus, whether an increase, decrease or no change in a biomarker level compared to a reference level indicates that the subject is likely to have slow progressing ALS or rapid progressing ALS or a particular progression rate will depend on whether the reference level is representative of a healthy subject, a subject with slow progressing ALS, a subject with rapid progressing ALS or a particular progression rate. For example, as described above and herein, subjects that have rapid progressing ALS typically have an increased (or elevated) level of sCD14 and/or LBP in a biological sample (e.g. blood, serum, plasma, CSF or urine) compared to subjects with slow progressing ALS and compared to healthy subjects. As also demonstrated herein, the levels of sCD14 and LBP strongly correlate with the rate of progression of ALS, wherein low levels of sCD14 and/or LBP correlate with lower rates of progression (e.g. as measured in AALS points per month) than do higher levels of sCD14 and/or LBP.

In instances where an assessment of the rate of progression is made by measuring the level of one or more ALS biomarkers, a direct comparison between the level of the biomarker and a reference level representative of a particular progression rate (e.g. as generated by a standard curve from a cohort of ALS patients with known progression rates) can be made, whereby if the level of the biomarker is the same or similar to the reference level, the progression rate of ALS in the subject is assessed as being the same or similar to the progression rate represented by that reference level.

In other examples, an increase in the level of sCD14 in a biological sample from a subject with ALS as compared to a reference level that is representative of a healthy subject or a subject with slow progressing ALS can indicate that the subject with ALS is likely to have rapid progressing ALS. If the reference level is representative of a subject with rapid progressing ALS, then a level of sCD14 that is the same or similar to the reference level would indicate that the subject has rapid progressing ALS. Conversely, as described herein, subjects that have slow progressing ALS typically have a decreased (or reduced) level of sCD14 compared to subjects with rapid progressing ALS. Thus, a decrease in the level of sCD14 in a biological sample from a subject with ALS as compared to a reference level that is representative of a subject with rapid progressing ALS indicates that the subject with ALS is likely to have slow progressing ALS. If the reference level is representative of a healthy subject or a subject with slow progressing ALS, then a level of sCD14 in a biological sample that is the same or similar to the reference level can indicate that the subject with ALS has slow progressing ALS.

Similarly, an increase in the level of LBP in a biological sample from a subject with ALS as compared to a reference level that is representative of a healthy subject or a subject with slow progressing ALS can indicates that the subject with LBP is likely to have rapid progressing ALS. If the reference level is representative of a subject with rapid progressing ALS, then a level of LBP that is the same or similar to the reference level would indicate that the subject has rapid progressing ALS. Conversely, as described herein, subjects that have slow progressing ALS typically have a decreased (or reduced) level of LBP compared to subjects with rapid progressing ALS. Thus, a decrease in the level of LBP in a biological sample from a subject with ALS as compared to a reference level that is representative of a subject with rapid progressing ALS indicates that the subject with ALS is likely to have slow progressing ALS. If the reference level is representative of a healthy subject or a subject with slow progressing ALS, then a level of LBP in a biological sample that is the same or similar to the reference level can indicate that the subject with ALS has slow progressing ALS.

In particular embodiments, the reference level is a threshold, above or below which is indicative of a subject having rapid progressing ALS or slow progressing ALS. For example, the reference level may be a threshold wherein a level of a biomarker in a sample that is equal to or above the reference level indicates that the subject is likely to have rapid progressing ALS, and a level of a biomarker in a sample that is below the reference level indicates that the subject is likely to have slow progressing ALS. In other examples, the reference level may be a threshold wherein a level of a biomarker in a sample that is above the reference level indicates that the subject is likely to have rapid progressing ALS, and a level of a biomarker in a sample that is equal to or below the reference level indicates that the subject is likely to have slow progressing ALS.

Thresholds may be selected that provide an acceptable ability to predict likelihood of a subject having slow or rapid progressing ALS. In illustrative examples, receiver operating characteristic (ROC) curves are calculated by plotting the value of a variable (e.g. a biomarker level) versus its relative frequency in two populations, e.g. in which a first population is considered likely to have rapid progressing ALS and a second population is considered likely to have slow progressing ALS.

For any particular biomarker, a distribution of biomarker levels for subjects who have particular rates of ALS progression, subjects who have or are likely to have rapid progressing ALS and subjects who have or are likely to have slow progressing ALS may overlap. Under such conditions, a test may not absolutely distinguish a subject with a particular rate of progression from a subject with another raye of progression, or a subject who is likely to have rapid progressing ALS from a subject who is likely to have slow progressing ALS, with absolute (i.e., 100%) accuracy, and the area of overlap indicates where the test cannot distinguish the two subjects. A threshold can be selected, above which (or below which, depending on how a biomarker changes with risk) the test is considered to be “positive” and below which the test is considered to be “negative.” The area under the ROC curve (AUC) provides the C-statistic, which is a measure of the probability that the perceived measurement will allow correct identification of a condition (see, e.g., Hanley et al., Radiology 143: 29-36 (1982)). Those skilled in the art can readily determine appropriate reference levels, including thresholds, for use in accordance with the presently disclosed methods.

### 3. Kits, Solid Supports and Compositions

The present invention also extends to kits for determining the level of one or more biomarkers. These kits can therefore be used to assess the rate of progression of ALS in a subject, or to determine the likelihood of a subject with ALS having slow or rapid progressing ALS. Such protein-based detection kits may include one or more antigen-binding molecules (e.g. an antibody or antigen-binding fragment thereof) that bind specifically to the one or more biomarkers described above and herein, and optionally at least one biomarker, which may be used as a positive control. In some embodiments, therefore, the kit comprises an antigen-binding molecule specific for sCD14 and/or an antigen-binding molecule specific for LBP. In further embodiments, the kit contains an antigen-binding molecule specific for at least one other biomarker, such as CRP, MIF, sTNFRI, sTNFRII, NFL, pNfH, p75NTR^{ECD}, miR-206, miR-143-3p, and/or miR-374b-5p. Thus, also contemplated is the use of an antigen-binding molecule specific for sCD14 and/or the use of an antigen-binding molecule specific for LBP for the preparation of a kit for assessing the rate of progression of ALS in a subject, or for determining the likelihood of a subject with ALS having slow or rapid progressing ALS. In some example, the use also includes the use of at least one other antigen-binding molecule specific for another biomarker, such as CRP, MIF, sTNFRI, sTNFRII, NFL, pNfH, p75NTR^{ECD}, miR-206, miR-143-3p, and/or miR-374b-5p. Antigen-binding molecules, such as antibodies and antigen-binding fragments thereof, that are specific for sCD14 or other biomarkers are well known in the art and can be used in the kits and uses described herein.

The kits may also comprise suitable detection agents, including for example, conjugates and substrates to facilitate detection (e.g. antibodies labelled with streptavidin, biotin, horseradish peroxidase etc.). The kit can also feature various devices and additional reagents and/or buffers, and/or printed instructional material for using the kit to quantify levels of one or more biomarkers. The kit reagents described herein, which may be optionally associated with detectable labels, can be presented in the format of a plurality of beads, a multiwall plate, a microarray, slide, microfluidics card, or a chip adapted for use with the techniques described herein for the measurement of biomarker levels in a sample.

Materials suitable for packing the components of the kits may include crystal, plastic (polyethylene, polypropylene, polycarbonate and the like), bottles, vials, paper, envelopes and the like. Additionally, the kits of the disclosure can contain instructional material for the simultaneous, sequential or separate use of the different components contained in the kit. The instructional material can be in the form of printed material or in the form of an electronic support capable of storing instructions such that they can be read by a subject, such as electronic storage media (magnetic disks, tapes and the like), optical media (CD-ROM, DVD) and the like. Alternatively or in addition, the media can contain Internet addresses that provide the instructional material.

Also provided are solid supports and compositions, such as for use in methods for assessing the rate of progression of ALS in a subject or for determining the level of one or more biomarkers, comprising one or more antigen-binding molecules (e.g. an antibody or antigen-binding fragment thereof) that bind specifically to the one or more biomarkers described above and herein. In a particular embodiment, the solid supports and/or compositions comprise an antigen-binding molecule specific for sCD14 and/or an antigen-binding molecule specific for LBP. The solid supports and compositions can optionally also comprise an antigen binding-molecule specific for at least one other biomarker, such as CRP, MIF, sTNFRI, sTNFRII, NFL, pNfH, p75NTR^{ECD}, miR-206, miR-143-3p, and/or miR-374b-5p. Exemplary solid supports include, but are not limited to, multiwell plates, slides and chips. In other embodiments, the solid support is a plurality of beads. As described above, these solid supports can be used in multiplex immunoassays to determine the level of two or more biomarkers in a biological sample, such as the level of sCD14 and/or LBP, and optionally one or more of CRP, MIF, sTNFRI, sTNFRII, NFL, pNfH, p75NTR^{ECD}, miR-206, miR-143-3p, and/or miR-374b-5p.

### 4. Subject Stratification and Therapeutic Applications

The present disclosure extends to subject stratification, such as for clinical trials or the management of disease or the treatment of disease, and also to therapeutic applications. For example, subjects that have rapid progressing ALS may be identified and selected for inclusion in clinical trials assessing the efficacy of new therapies. The inclusion of such subjects in clinical trials is desirable because the shorter time period for disease progression can yield more rapid evidence of efficacy of the therapy. In other embodiments, appropriate or optimized treatment regimens can be devised based on whether a subject is identified as being likely to have slow or rapid progressing ALS.

Treatment regimens described as being useful for the treatment of ALS include those that comprise administration of one or more anti-neurodegenerative agents. These agents include, but are not limited to, riluzole (Rilutek®/Teglutik®), edaravone (Radicava®/Radicut®), a CD14 antagonist (e.g. a CD14 anatagonist antibody), anti-inflammatories, e.g. blockers of the complement pathway, such as C5a receptor agonists (e.g. PMX205 or eculizumab (Lee J. D. et al., (2017) British Journal of Pharmacology, 174(8)), an agent that blocks the interaction between CD40 and CD40 ligand (including antibodies that bind specifically to CD40 and/or CD40 ligand (e.g., AT-1502), GM604, and masitinib.

In one embodiment, the treatment regimen includes administration, such as systemic (e.g. intravenous) administration of a CD14 antagonist antibody. The CD14 antagonist antibody can bind to CD14 (e.g. mCD14 or sCD14) and block the binding of a DAMP or PAMP to CD14 and/or bind to CD14 and inhibit or decrease a CD14 agonist-mediated response resulting in the production of pro-inflammatory mediators, including the production of pro-inflammatory cytokines. In some embodiments, the CD14 antagonist antibody inhibits binding of a CD14 agonist, suitably a DAMP or PAMP, to CD14 thus inhibiting or decreasing the production of pro-inflammatory cytokines. In illustrative examples of this type, the CD14 antagonist antibody is selected from the 3C10 antibody that binds an epitope comprised in at least a portion of the region from amino acid 7 to amino acid 14 of human CD14 (van Voohris et al., 1983. J. Exp. Med. 158: 126-145; Juan et al., 1995. J. Biol. Chem. 270(29): 17237-17242), the MEM-18 antibody that binds an epitope comprised in at least a portion of the region from amino acid 57 to amino acid 64 of CD14 (Bazil et al., 1986. Eur. J. Immunol. 16(12):1583-1589; Juan et al., 1995. J. Biol. Chem. 270(10): 5219-5224), the 4C1 antibody (Adachi et al., 1999. J. Endotoxin Res. 5: 139-146; Tasaka et al., 2003. Am. J. Respir. Cell. Mol. Biol.; 2003. 29(2):252-258), as well as the 28C5 and 23G4 antibodies that inhibit binding of LPS and suppress production of pro-inflammatory cytokines, and the 18E12 antibody that partly inhibits binding of LPS and suppresses production of pro-inflammatory cytokines (U.S. Pat. Nos. 5,820,858, 6,444,206 and 7,326,569). In some embodiments, a CD14 antagonist antibody of the present disclosure inhibits binding of CD14 to a TLR such as TLR4, thereby blocking CD14-agonist mediated response, illustrative examples of which include the F1024 antibody disclosed in International Publication WO2002/42333. The CD14 antagonist antibody may be a full-length immunoglobulin antibody or an antigen-binding fragment of an intact antibody, representative examples of which include a Fab fragment, a F(ab′)2 fragment, an Fd fragment consisting of the VH and CH1 domains, an Fv fragment consisting of the VL and VH domains of a single arm of an antibody, a single domain antibody (dAb) fragment (Ward et al., 1989. Nature 341:544-546), which consists of a VH domain; and an isolated CDR. Suitably, CD14 antagonist antibody is a chimeric, humanized or human antibody.

In some embodiments, the CD14 antagonist antibody is selected from the antibodies disclosed in U.S. Pat. No. 5,820,858:
(1) an antibody comprising:
   a VL domain comprising, consisting or consisting essentially of the sequence:

[SEQ ID NO: 1]
QSPASLAVSLGQRATISC RASESVDSFGNSFMH WYQQKAGQPPKSSIY
RAANLESGIPARFSGSGSRTDFTLTINPVEADDVATYFC QQSYEDPWT
FGGGTKLGNQ (3C10 VL);

and
a VH domain comprising, consisting or consisting essentially of the sequence:

[SEQ ID NO: 2]
LVKPGGSLKLSCVASGFTFS SYAMS WVRQTPEKRLEWVA
SISSGGTTYYPDNVKG RFTISRDNARNILYLQMSSLRSEDTAMYYCAR
GYYDYHY WGQGTTLTVSS (3C10 VH);

(2) an antibody comprising:
   a VL domain comprising, consisting or consisting essentially of the sequence:

[SEQ ID NO: 3]
QSPASLAVSLGQRATISC RASESVDSYVNSFLH WYQQKPGQPPKLLIY
RASNLQSGIPARFSGSGSRTDFTLTINPVEADDVATYCC QQSNEDPTT
FGGGTKLEIK (28C5 VL);

and
a VH domain comprising, consisting or consisting essentially of the sequence:

[SEQ ID NO: 4]
LQQSGPGLVKPSQSLSLTCTVTGYSIT SDSAWN WIRQFPGNRLEWMG
YISYSGSTSYNPSLKS RISITRDTSKNQFFLQLNSVTTEDTATYYCVR
GLRFAY WGQGTLVTVSA (28C5 VH);

and
(3) an antibody comprising:
   a VL domain comprising, consisting or consisting essentially of the sequence:

[SEQ ID NO: 5]
QTPSSLSASLGDRVTISC RASQDIKNYLN WYQQPGGTVKVLIY
YTSRLHS GVPSRFSGSGSGTDYSLTISNLEQEDFATYFC
QRGDTLPWT FGGGTKLEIK (18E12 VL);

and
a VH domain comprising, consisting or consisting essentially of the sequence:

[SEQ ID NO: 6]
LESGPGLVAPSQSLSITCTVSGFSLT NYDIS WIRQPPGKGLEWLG
VIWTSGGTNYNSAFMS RLSITKDNSESQVFLKMNGLQTDDTGIYYCVR
GDGNFYLYNFDY WGQGTTLTVSS (18E12 VH);

Also contemplated are antibodies that comprise the VL and VH CDR sequences of the above antibodies, representative embodiments of which include:
(1) an antibody that comprises: a) an antibody VL domain, or antigen binding fragment thereof, comprising L-CDR1, L-CDR2 and L-CDR3, wherein: L-CDR1 comprises the sequence RASESVDSFGNSFMH [SEQ ID NO: 7] (3C10 L-CDR1); L-CDR2 comprises the sequence RAANLES [SEQ ID NO: 8] (3C10 L-CDR2); and L-CDR3 comprises the sequence QQSYEDPWT [SEQ ID NO: 9] (3C10 L-CDR3); and b) an antibody VH domain, or antigen binding fragment thereof, comprising H-CDR1, H-CDR2 and H-CDR3, wherein: H-CDR1 comprises the sequence SYAMS [SEQ ID NO: 10] (3C10 H-CDR1); H-CDR2 comprises the sequence SISSGGTTYYPDNVKG [SEQ ID NO: 11] (3C10 H-CDR2); and H-CDR3 comprises the sequence GYYDYHY [SEQ ID NO: 12] (3C10 H-CDR3);
(2) an antibody that comprises: a) an antibody VL domain, or antigen binding fragment thereof, comprising L-CDR1, L-CDR2 and L-CDR3, wherein: L-CDR1 comprises the sequence RASESVDSYVNSFLH [SEQ ID NO: 13] (28C5 L-CDR1); L-CDR2 comprises the sequence RASNLQS [SEQ ID NO: 14] (28C5 L-CDR2); and L-CDR3 comprises the sequence QQSNEDPTT [SEQ ID NO: 15] (28C5 L-CDR3); and b) an antibody VH domain, or antigen binding fragment thereof, comprising H-CDR1, H-CDR2 and H-CDR3, wherein: H-CDR1 comprises the sequence SDSAWN [SEQ ID NO: 16] (28C5 H-CDR1); H-CDR2 comprises the sequence YISYSGSTSYNPSLKS [SEQ ID NO: 17] (28C5 H-CDR2); and H-CDR3 comprises the sequence GLRFAY [SEQ ID NO: 18] (28C5 H-CDR3); and
(3) an antibody that comprises: a) an antibody VL domain, or antigen binding fragment thereof, comprising L-CDR1, L-CDR2 and L-CDR3, wherein: L-CDR1 comprises the sequence RASQDIKNYLN [SEQ ID NO: 19] (18E12 L-CDR1); L-CDR2 comprises the sequence YTSRLHS [SEQ ID NO: 20] (18E12 L-CDR2); and L-CDR3 comprises the sequence QRGDTLPWT [SEQ ID NO: 21] (18E12 L-CDR3); and b) an antibody VH domain, or antigen binding fragment thereof, comprising H-CDR1, H-CDR2 and H-CDR3, wherein: H-CDR1 comprises the sequence NYDIS [SEQ ID NO: 22] (18E12 H-CDR1); H-CDR2 comprises the sequence VIWTSGGTNYNSAFMS [SEQ ID NO: 23] (18E12 H-CDR2); and H-CDR3 comprises the sequence GDGNFYLYNFDY [SEQ ID NO: 24] (18E12 H-CDR3).

In some embodiments, the CD14 antagonist antibody is humanized. In illustrative examples of this type, the humanized CD14 antagonist antibodies suitably comprise a donor CDR set corresponding to a CD14 antagonist antibody (e.g., one of the CD14 antagonist antibodies described above), and a human acceptor framework. The human acceptor framework may comprise at least one amino acid substitution relative to a human germline acceptor framework at a key residue selected from the group consisting of: a residue adjacent to a CDR; a glycosylation site residue; a rare residue; a canonical residue; a contact residue between heavy chain variable region and light chain variable region; a residue within a Vernier zone; and a residue in a region that overlaps between a Chothia-defined VH CDR1 and a Kabat-defined first heavy chain framework. Techniques for producing humanized mAbs are well known in the art (see, for example, Jones et al., 1986. Nature 321: 522-525; Riechmann et al. 1988. Nature 332:323-329; Verhoeyen et al., 1988. Science 239: 1534-1536; Carter et al., 1992. Proc. Natl. Acad. Sci. USA 89: 4285-4289; Sandhu, J S., 1992. Crit. Rev. Biotech. 12: 437-462, and Singer et al., 1993. J. Immunol. 150: 2844-2857). A chimeric or murine monoclonal antibody may be humanized by transferring the mouse CDRs from the heavy and light variable chains of the mouse immunoglobulin into the corresponding variable domains of a human antibody. The mouse framework regions (FR) in the chimeric monoclonal antibody are also replaced with human FR sequences. As simply transferring mouse CDRs into human FRs often results in a reduction or even loss of antibody affinity, additional modification might be required in order to restore the original affinity of the murine antibody. This can be accomplished by the replacement of one or more human residues in the FR regions with their murine counterparts to obtain an antibody that possesses good binding affinity to its epitope. See, for example, Tempest et al. (1991. Biotechnology 9:266-271) and Verhoeyen et al. (1988 supra). Generally, those human FR amino acid residues that differ from their murine counterparts and are located close to or touching one or more CDR amino acid residues would be candidates for substitution.

In one embodiment, the CD14 antagonist antibody is the IC14 antibody (Axtelle et al., 2001. J. Endotoxin Res. 7: 310-314; and U.S. Pat. Appl. No. 2006/0121574, which are incorporated herein by reference in their entirety) or an antigen-binding fragment thereof. The IC14 antibody is a chimeric (murine/human) monoclonal antibody that specifically binds to human CD14. The murine parent of this antibody is 28C5 noted above (see, U.S. Pat. Nos. 5,820,858, 6,444,206 and 7,326,569 to Leturcq et al., and Leturcq et al., 1996. J. Clin. Invest. 98: 1533-1538). The IC14 antibody comprises a VL domain and a VH domain, wherein:
the VL domain comprises the amino acid sequence:

[SEQ ID NO: 25]
METDTILLWVLLLWVPGSTGDIVLTQSPASLAVSLGQRATISCRASESVD
SYVNSFLHWYQQKPGQPPKLLIYRASNLQSGIPARFSGSGSRTDFTLTIN
PVEADDVATYYCQQSNEDPYTFGGGTKLEIKRTVAAPSVFIFPPSDEQLK
SGTASVVCLLNNFYPREAKVQWKVDNALQSGNSQESVTEQDSKDSTYSLS
STLTLSKADYEKHKVYACEVTHQGLSSPVTKSFNRGEC;

and
the VH domain comprises the amino acid sequence:

[SEQ ID NO: 26]
MKVLSLLYLLTAIPGILSDVQLQQSGPGLVKPSQSLSLTCTVTGYSITSD
SAWNWIRQFPGNRLEWMGYISYSGSTSYNPSLKSRISITRDTSKNQFFLQ
LNSVTTEDTATYYCVRGLRFAYWGQGTLVTVSSASTKGPSVFPLAPCSRS
TSESTAALGCLVKDYFPEPVTVSWNSGALTSGVHTFPAVLQSSGLYSLSS
VVTVPSSSLGTKTYTCNVDHKPSNTKVDKRVESKYGPPCPSCPAPEFLGG
PSVFLFPPKPKDTLMISRTPEVTCVVVDVSQEDPEVQFNWYVDGVEVHNA
KTKPREEQFNSTYRVVSVLTVLHQDWLNGKEYKCKVSNKGLPSSIEKTIS
KAKGQPREPQVYTLPPSQEEMTKNQVSLTCLVKGFYPSDIAVEWESNGQP
ENNYKTTPPVLDSDGSFFLYSRLTVDKSRWQEGNVFSCSVMHEALHNHYT
QKSLSLSLGK.

Two or more anti-neurodegenerative agents may be used in combination for their additive activity and, in certain instances, for their synergistic effects. When combination therapy is desired, the agents can be administered separately, simultaneously or sequentially. In some embodiments, this may be achieved by administering a single composition or pharmacological formulation that includes each of the agents, or by administering two or more separate compositions or formulations (each containing one or more agents) at the same time. In other embodiments, the treatment with one agent may precede or follow the treatment with the other agent by intervals ranging from minutes to days.

Where two or more agents are administered to a subject “in conjunction” or “concurrently” they may be administered in a single composition at the same time, or in separate compositions at the same time, or in separate compositions separated in time.

The treatment regimen may also, or alternatively, comprise other interventions. These include medicinal and/or nutritional interventions such as provided via food, a tablet, oral solution, patch or intravenous injection or other parenteral mode of administration. In other examples, the intervention may be mechanical such as a non-invasive ventilation device that is used to ease breathing difficulties. Non-invasive ventilation may include, for example, Negative Pressure Ventilation (NPV) and/or Noninvasive Positive Pressure Ventilation (NIPPV), examples of the latter being Continuous Positive Airway Pressure (CPAP), Bilevel Positive Airway Pressure (BiPAP), and Average Volume Assured Pressure Support (AVAPS). The subject methods may also be practiced in combination with other therapy, including but not limited to physical therapy, speech therapy, psychotherapy and occupational therapy.

The anti-neurodegenerative agents may be administered in an “effective amount (s)”, to achieve an intended purpose in a subject, such as the alleviation of symptoms associated with ALS. The dose of agents(s) administered to a patient should be sufficient to achieve a beneficial response in the subject over time such as a reduction in at least one symptom associated with a neurodegenerative disease. In some embodiments, there is a reduction in at least one symptom selected from progressive muscle atrophy, paralysis, spasticity, hyperreflexia, respiratory function and other symptoms such as difficulty swallowing, limb weakness, dysarthria, slurred speech, impaired gait, facial weakness and muscle cramps.

The quantity or dose frequency of the agent(s) to be administered may depend on the subject to be treated inclusive of the age, sex, weight and general health condition, burden of disease and progression rate of disease. In this regard, precise amounts of the agent(s) for administration will depend on the judgment of the practitioner. One skilled in the art would be able, by routine experimentation, to determine an effective amount of an anti-neurodegenerative agent described herein, to include in a pharmaceutical composition for the desired therapeutic outcome.

In a particular embodiment, the treatment regimen to which the subject is exposed does not comprise the administration of one or more anti-neurodegenerative agents. Such treatment regimens may instead involve the administration of nutritional supplements and/or the development of an appropriate diet, non-invasive ventilation, physical therapy, speech therapy, psychotherapy and/or occupational therapy. For example, in instances where a subject is determined to be likely to have slow progressing ALS, a treatment regimen that does not comprise the administration of one or more neurodegenerative agents, or does not comprise the administration of one or more neurodegenerative agents for at least a particular time period, such as 6, 12, 18, 24, 30, 36 or more months, is implemented. In other examples, when the subject is determined to be likely to have rapid progressing ALS, the subject may be put on non-invasive ventilation earlier than subjects with slow progressing ALS.

In order that the invention may be readily understood and put into practical effect, particular preferred embodiments will now be described by way of the following non-limiting examples.

### EXAMPLES

### Example 1

### Study Subjects

Standard protocol approvals, registrations, and patient consents. This was a prospective cohort study in which serum samples were collected from patients with ALS and health volunteers (HV; also referred to as healthy controls, controls or NC) from the MDA/ALSA ALS clinic at the Houston Methodist Hospital. Written informed consent was obtained from all participants following ethics approval from the Institutional Review Board (IRB) at Houston Methodist Hospital. Patients with ALS (recruited between January 2011 and November 2016) were diagnosed according to the revised El Escorial criteria and the Appel ALS (AALS) score (range: 30-164, Haverkamp et al., 1995 Brain 118:707-719; Voustianiouk et al., 2008 Muscle Nerve. 37(5):668-72) by an experienced ALS neurologist (SHA). None of the patients with ALS had ongoing infectious diseases. HV (recruited between June 2008 and February 2015) were typically spouses and friends of patients, and exclusion criteria included any neurologic condition, auto immune diseases, or infectious diseases. Clinical information was collected by the investigator from symptom onset and diagnosis to baseline assessment and sample collection. The demographics between the ALS patients and volunteers were similar.

### Quantitation of Biomarkers

sCD14, LBP, CRP, MIF, sTNFRI and sTNFRII were quantitated by ELISA.

### Monocyte Isolation

Human monocytes were freshly isolated from peripheral blood of ALS patients and normal controls. A human pan monocyte isolation kit (Miltenyi Biotec, San Diego, Calif., USA) was used to obtain highly pure pan monocytes by negative selection according to manufacturer's instructions.

### Flow Cytometry

Isolated pan monocytes were stained with Fc blocker to avoid non-specific binding. Both isolated monocytes and fresh blood samples were then incubated with anti-human CD14-V450 antibody (eBioscience, San Diego, Calif., USA), anti-human CD16-FITC (eBioscience, San Diego, Calif., USA), anti-human HLA-DR-PerCP Cy5.5 (eBioscience, San Diego, Calif., USA), anti-human TIM3-PE (eBioscience, San Diego, Calif., USA). One additional lysing step to remove red blood cells was done for peripheral blood samples. Dead cells were stained using LIVE/DEAD® Fixable Blue Dead Cell Stain Kit (Molecular Probes, Eugene, Oreg., USA). The cells were immediately analyzed using an LSR II™ 13 color flow cytometer configured with 355, 488, 405, 561, and 633 lasers.

### Statistics

Comparisons were performed using ANOVA for more than 2 groups or Student's t-test for two groups. Correlation was done using Spearman Rank Order in SigmaStat software. ROC curve analysis was performed using the R statistical software package (Vienna, Austria; V.3.0.2). Data are expressed as mean±SE and p values less than 0.05 were considered significant.

### Results

CD14 and CD16 Surface Markers on PBMC and Isolated Pan Monocytes from Patients with ALS and HV.

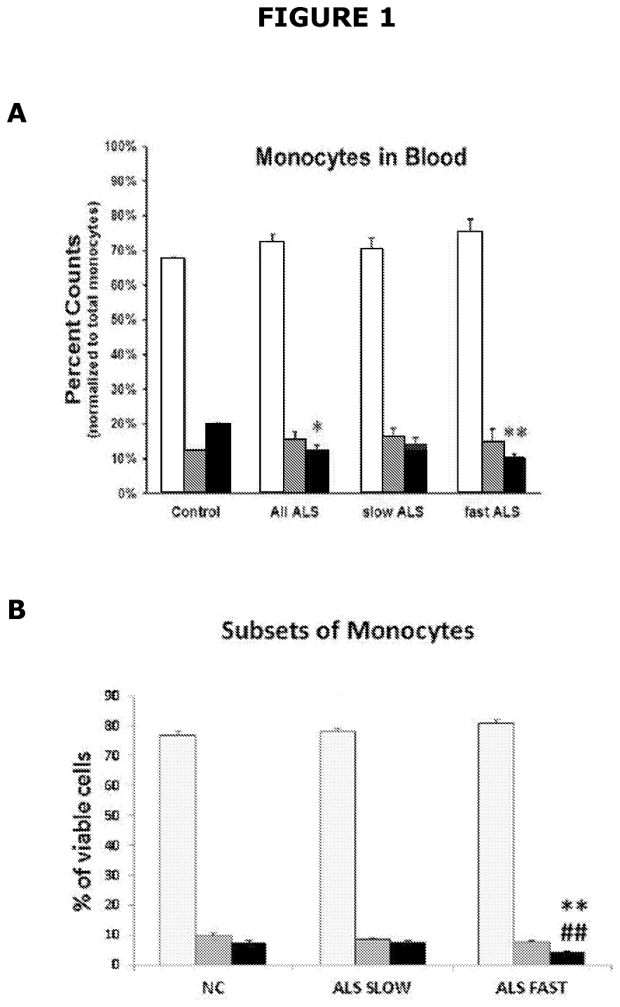

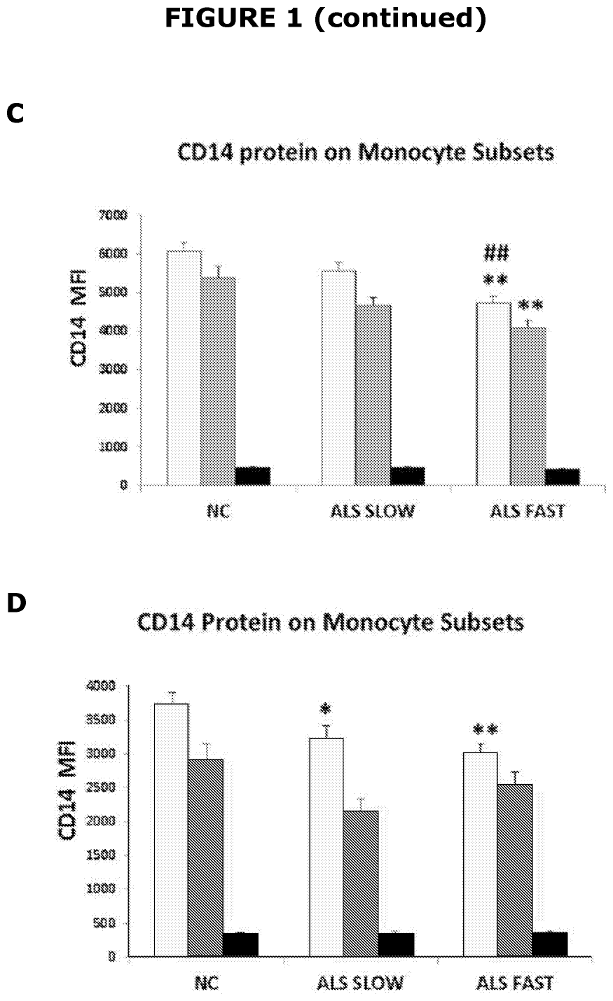

CD14 and CD16 are expressed predominately by monocytes/macrophages. Based on their cell surface expression, human monocytes can be divided into three distinct subsets: CD14+/CD16−, CD14+/CD16+, and CD14−/low/CD16+ monocytes. Using flow cytometry, CD14 and CD16 monocyte surface markers were quantitated on PBMC from a cohort of patients with ALS and age-matched HV (FIG. 1A). The frequency of CD14−/low/CD16+ monocytes were decreased in the total PBMC samples of all patients with ALS compared with HV (p=0.04). Patients with ALS were then separated into rapid progressing patients, defined as those progressing at a rate of greater than or equal to 1.5 Appel ALS (AALS scoring system; Haverkamp et al., Brain 1995; 118:707-719) points/month, and slow progressing patients, defined as progressing at a rate of less than 1.5 AALS points/month (Henkel et al., EMBO Mol Med 2013; 5:64-79). Rapid progressing patients had reduced numbers of CD14−/low/CD16+ monocytes in their PBMC (p=0.016). No differences were observed in the frequencies of the CD14+/CD16− and CD14+/CD16+ populations.

In a second cohort of patients with ALS and age-matched HV, pan monocytes were isolated and purified from PBMC using a negative selection protocol to avoid possible monocyte activation, and then again, subjected to flow cytometric analyses; positive selection or lengthy gradient separation may activate monocytes. The frequency of the CD14−/low/CD16+ subset of purified monocytes was again decreased in rapid progressing patients compared with slow progressing patients (p<0.001) and HV (p<0.001) (FIG. 1B). As was observed in the PBMC samples, no differences were noted in the frequencies of the CD14+/CD16− and CD14+/CD16+ populations.

To verify that cell surface expression of membrane-bound CD14 (mCD14) was indeed decreased, the CD14 median fluorescence intensity (MFI) was measured on the PBMC samples. The results demonstrated that patients with rapid (p<0.01) and slow progressing (p<0.01) ALS had reduced CD14 protein signal on surface of their CD14+/CD16− monocytes than HV (FIG. 1C). When the isolated and purified pan monocyte data were analyzed, the cell surface CD14 MFI was decreased on CD14+/CD16− and CD14+/CD16+ monocytes from patients with rapid progressing ALS compared with slow progressing patients with ALS (p<0.01) and HV (p<0.01) (FIG. 1D). These data suggest that frequency of CD14−/low/CD16+ monocytes and cell surface expression of CD14 are reduced on monocytes from patients either by actively shedding CD14 from the cell surface or by the reduced production and subsequent expression of CD14 on the monocyte cell surface.

### Correlation Between CD14−/Low/CD16+ Monocytes and ALS Disease Burdens or Rates of Progression

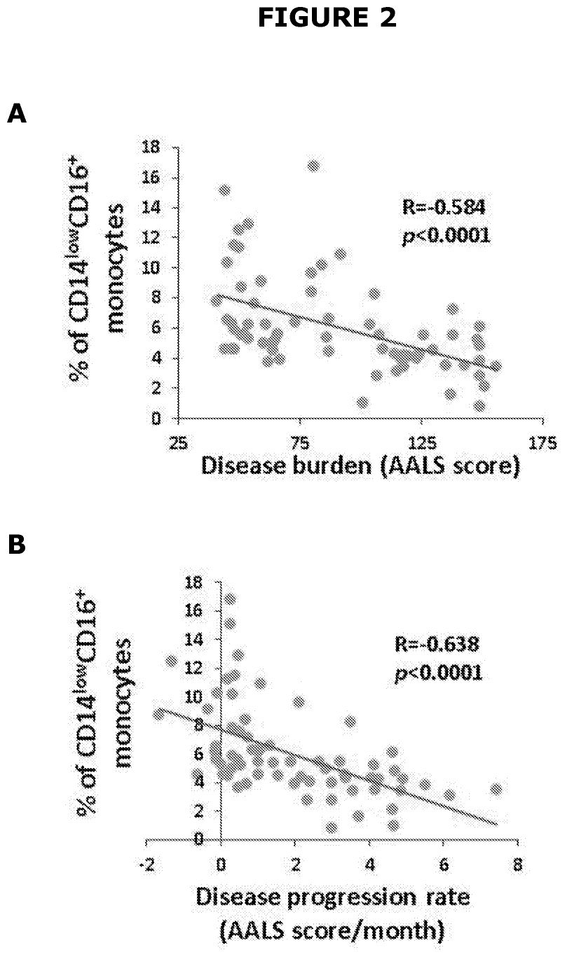

Since frequency of CD14−/low/CD16+ monocytes were demonstrated to be reduced in rapid progressing patients, Spearman's correlation coefficient analyses were performed to determine if the frequency of this subpopulation of monocytes is associated with either disease burdens or the rates of disease progression in patients with ALS. In patients with ALS, the percentage of CD14−/low/CD16+ monocytes was negatively correlated with disease burden based on the AALS scoring system (p<0.0001, R=0.584) (FIG. 2A); with greater disease burden, there was a reduced frequency of these monocytes. In addition, the percentage of CD14−/low/CD16+ monocytes was negatively correlated with rates of disease progression based on the AALS scoring system (p<0.0001, R=0.638) (FIG. 2B); the more rapid the disease progression rate, the greater the reduction of these monocytes.

The CD14−/Low/CD16+/TIM-3+ Subpopulation in CD14−/Low/CD16+ Cells in PBMC and Isolated Monocytes from Patients with ALS and HV.

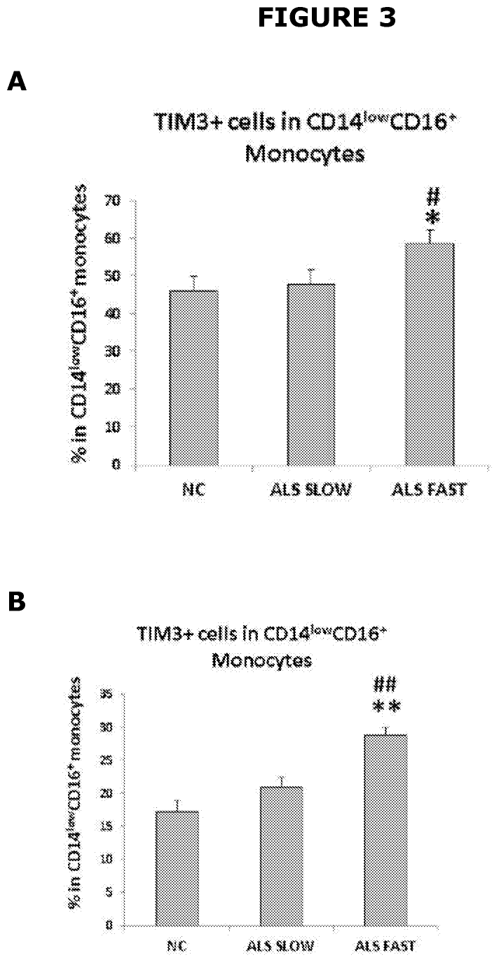

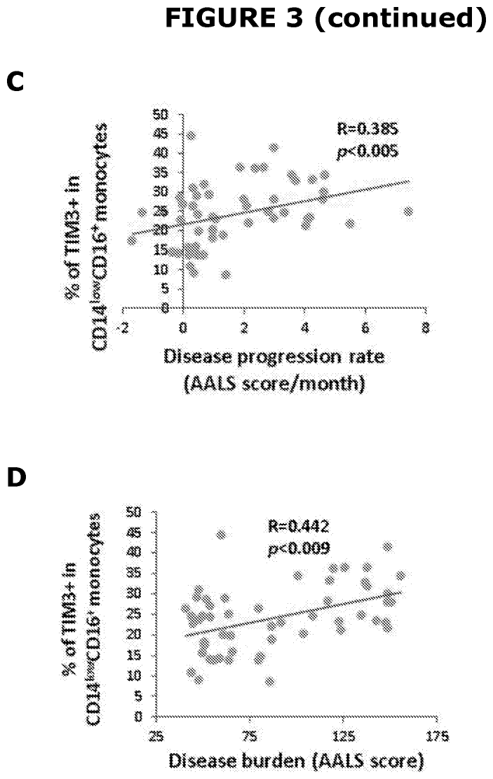

Since peripheral immune activation is now a recognized component of ALS pathology, and since TIM-3 has been shown to promote a pro-inflammatory response when expressed by innate immune cells (Anderson et al., Science 2007; 318(5853):1141-1143; Han et al., Front Immunol 2013; 4:449)), TIM-3 expression was used as a biomarker for the pro-inflammatory capacity of monocytes derived from patients with ALS. The frequency of CD14−/low/CD16+/TIM-3+ monocytes was increased in PBMC of rapid progressing patients with ALS compared with the frequency of CD14−/low/CD16+/TIM-3+ monocytes from slow progressing patients (p<0.001) and HV (p<0.001) (FIG. 3A). When isolated and purified pan monocytes were analyzed for their expression of TIM-3 on CD14−/low/CD16+, the frequency of CD14−/low/CD16+/TIM-3+ monocytes was again increased in rapid progressing patients compared with the frequency of CD14−/low/CD16+/TIM-3+ monocytes from slow progressing patients (p<0.001) and HV (p<0.001) (FIG. 3B). In both the PBMC and pan monocyte samples, frequencies of TIM-3 expression on CD14+CD16− or CD14+CD16+ monocytes were not changed from slow or rapid progressing patients or HV. The percentage CD14−/low/CD16+/TIM-3+ monocytes also positively correlated with disease progression rates and disease burden in patients with ALS (p=0.005, R=0.385; p=0.009, R=0.442, respectively) (FIGS. 3C and D).

Serum soluble CD14

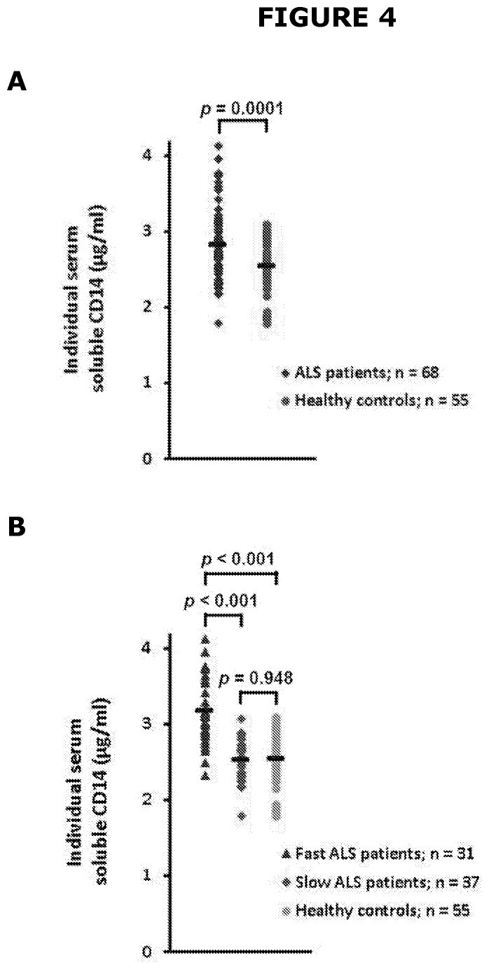

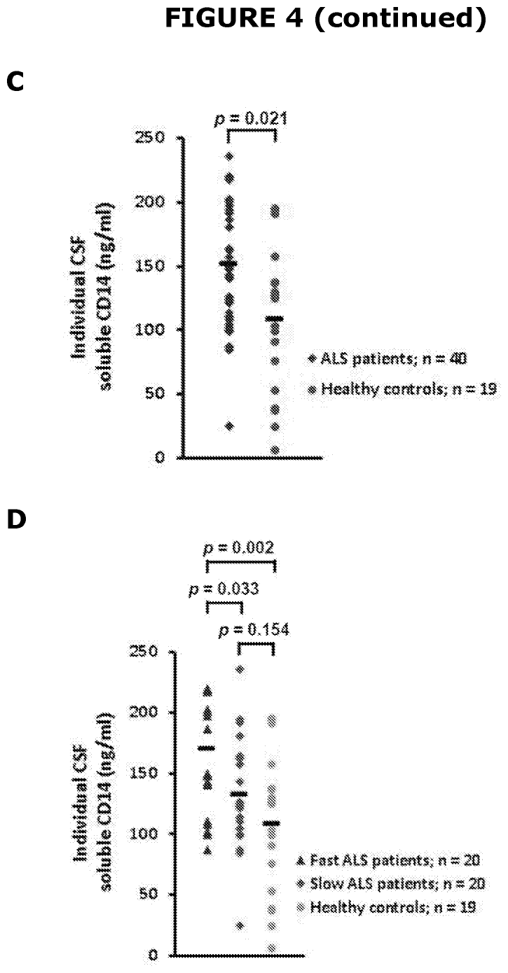

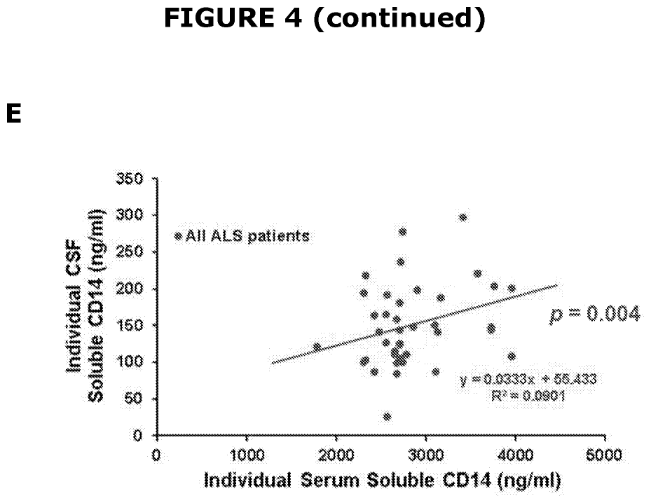

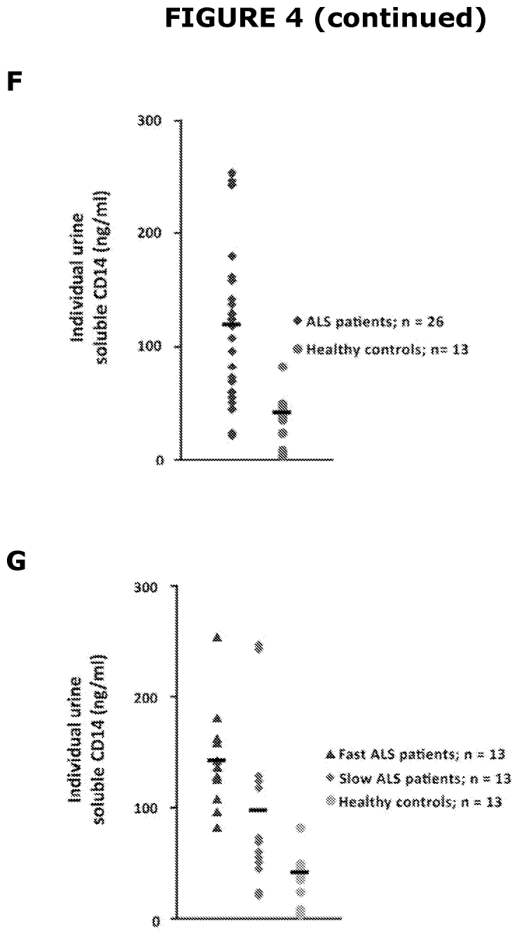

Upon monocyte activation, mCD14 is cleaved from the cell surface and soluble CD14 (sCD14) is released; increased serum sCD14 levels are considered a biomarker for monocyte activation (Shive C L et al., AIDS. 201; 29(10):1263-1265). Because the frequency of CD14−/low/CD16+ monocytes and monocyte surface expression of CD14 were reduced, sera were examined for sCD14 levels. Patient serum sCD14 levels were elevated compared with serum from HV (p=0.0001) (FIG. 4A). When further separated into rapid progressing and slow progressing patients (n=37), sCD14 was only elevated in serum from rapid progressing patients compared with either slow progressing patients (p<0.001) or HV (p<0.001) (FIG. 4B). There was no difference in serum sCD14 levels between slow progressing patients and HV (p=0.948).

sCD14 levels were also evaluated in the cerebrospinal fluid (CSF) and urine in a subpopulation of these patents. The absolute levels of CSF sCD14 were 10-fold less than that measured in the sera of these patients. sCD14 was elevated in the CSF of patients with ALS compared with CSF obtained from HV (p=0.021) (FIG. 4C). However, when patients were separated into rapid and slow progressing patients, only the CSF samples from rapid progressing patients had increased levels of sCD14 compared with slow progressing patients (p=0.033) and patients with HV (p=0.002); there were no difference between slow progressing patients with ALS and HV (p=0.154) (FIG. 4D). Furthermore, CSF levels of sCD14 positively correlated with serum levels of sCD14 (p=0.004, R=0.619) (FIG. 4E).

In relation to relation to levels of sCD14 in urine, sCD14 was elevated in patients with ALS compared with HV (p=0.000003) (FIG. 4F). When patients were separated into rapid and slow progressing patients, the urine samples from rapid progressing patients had increased levels of sCD14 compared with patients with HV (p=0.0000006), and there was also a trend towards increased levels of sCD14 in the urine of rapid progressing patients compared to slow progressing patients (p=0.077) (FIG. 4G). There was also a statistically significant increase in levels of urine sCD14 in slow progressing patients with ALS compared to HV, although this increase was less marked than seen with rapid progressing patients (p=0.022) (FIG. 4G).

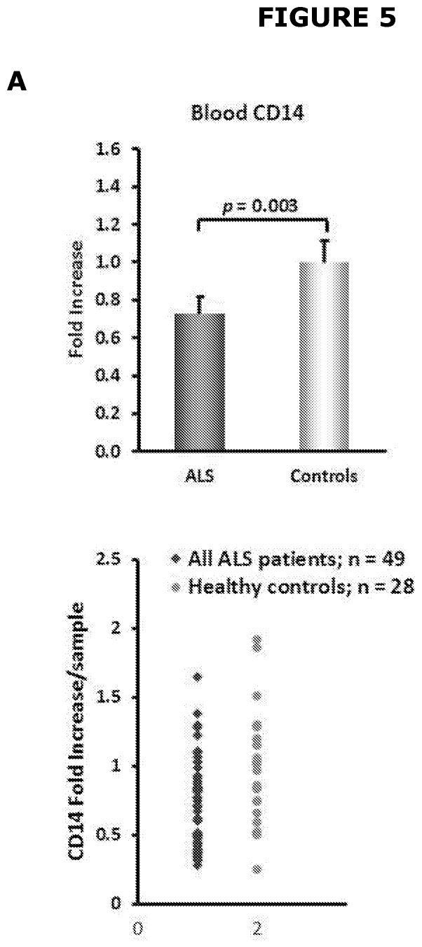

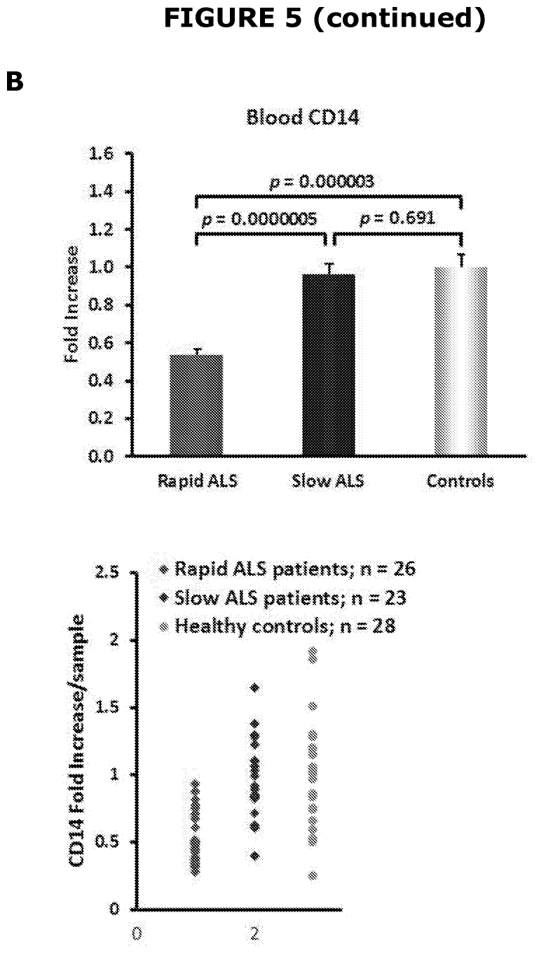

sCD14 can be generated either by cleavage from the cell surface or released from intracellular pools. To determine if CD14 mRNA is also decreased, qRT-PCR assays were performed on mRNA isolated from PBMC of patients and compared with mRNA isolated from HV. The CD14 mRNA was reduced in PBMC from patients (p=0.003) (FIG. 5A). When the mRNA samples were also separated into rapid progressing patients and slow progressing patients, CD14 mRNA was only reduced in the rapid progressing patients compared with either slow progressing patients (p<0.001) or HV (p<0.001) (FIG. 5B). There was no difference in CD14 mRNA between slow progressing patients with ALS and HV (p=0.691).

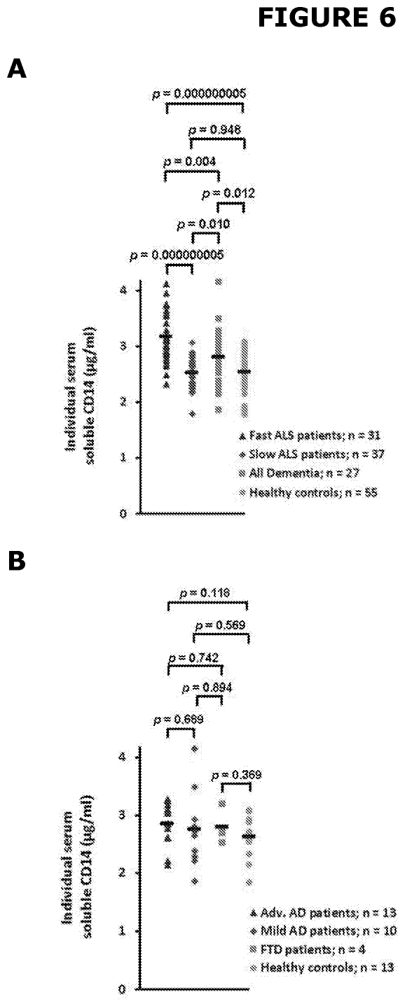

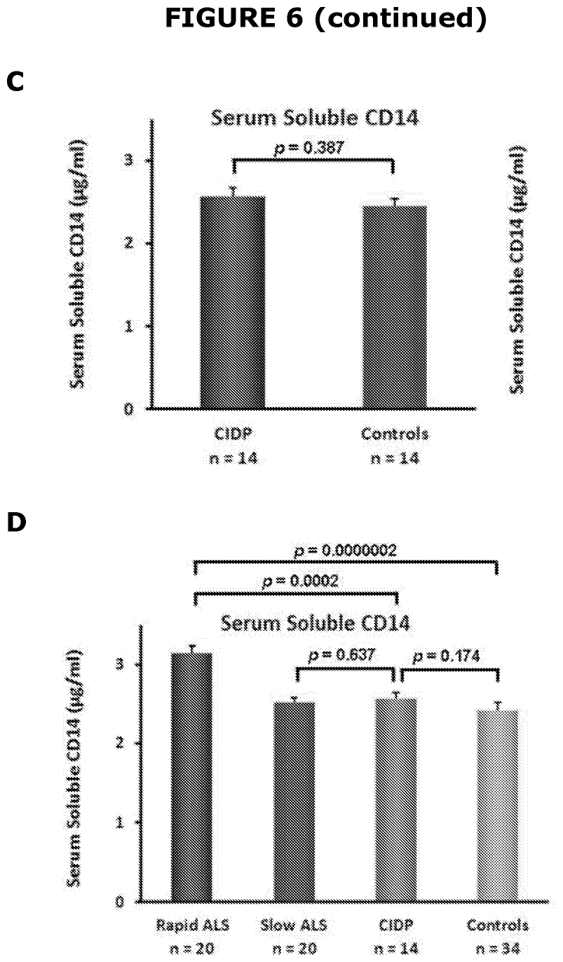

To determine if the increased serum sCD14 levels from patients with ALS were specific for this neurodegenerative disease, serum from patients with dementia (Alzheimer's disease and frontal temporal dementia), other neurodegenerative diseases, were assayed for their sCD14 concentrations and compared with appropriate age-matched HV (n=13). There were no differences in the serum levels of sCD14 from patients with dementia compared with HV (p=0.177) (FIG. 6A). When patients with dementia were separated into patients with mild Alzheimer's disease (Mild AD; n=10), patients with advanced Alzheimer's disease (AD; n=13), and patients with frontal temporal dementia (FTD; n=4), there were no difference among the different groups of patients (Mild AD vs. AD, p=0.689; Mild AD vs. FTD, p=0.894; and AD vs. FTD, p=0.742), and between each group of patients with dementia and HV (Mild AD vs. HV, p=0.569; AD vs. HV, p=0.118; and FTD vs. HV, p=0.369) (FIG. 6B). Patients with dementia and their age-matched HV were not compared directly with patients with ALS and their respective age-matched HV because patients with dementia/HV were older than patients with ALS/HV (data not shown).

To determine if the increased serum sCD14 levels from patients with ALS were different from another neurological disease, serum from patients with chronic inflammatory demyelinating polyneuropathy (CIDP, n=14), an autoimmune neurological disorder, were assayed for their sCD14 levels and compared with appropriate age-matched HV. Serum sCD14 levels were not different between patients with CIDP and HV (p=0.387) (FIG. 6C). In this analysis, the ages of patients with CIDP and their age-matched HV were not different than patients with ALS and their respective age-matched control, therefore, patients with CIDP were compared with patients with ALS (FIG. 6D). In addition, since the ages between the two groups of HV were not different (p=0.751), the serum sCD14 levels between the two groups were compared, and since there was not a difference (p=0.555), the two HV groups were combined (n=34). Serum sCD14 levels from all patients with ALS were increased compared with serum sCD14 levels from patients with CIDP (p=0.043). However, when patients with ALS were separated into rapid progressing (n=20) and slow progressing patients (n=20), and then compared with patients with CIDP, only the rapid progressing patients with ALS were different from patients with CIDP (p=0.0002); no differences were found between slow progressing patients with ALS and patients with CIDP (p=0.637). Furthermore, there were not a differences between patients with CIDP and the combined groups of HV (p=0.174).

Correlation Between Serum sCD14 and ALS Disease Burden

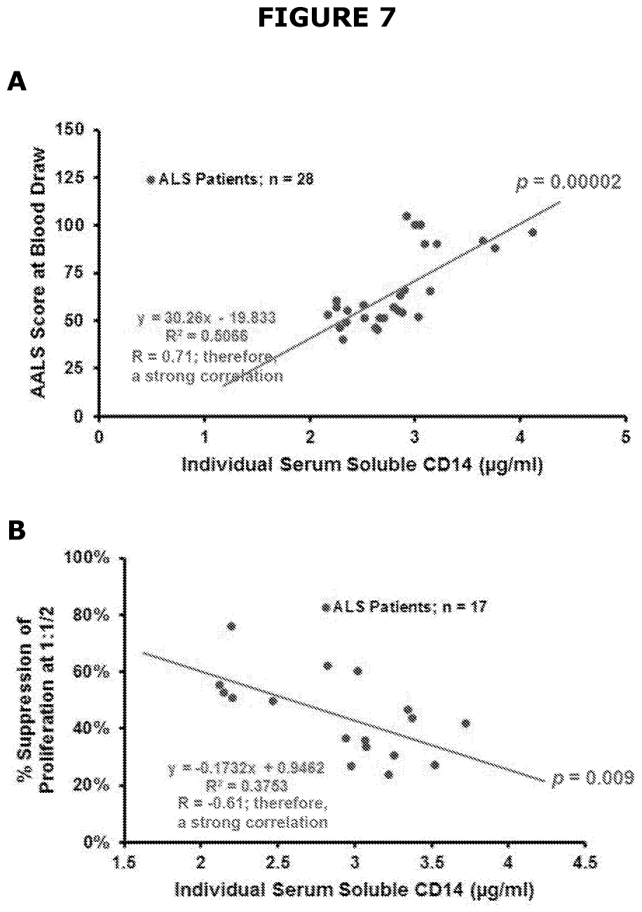

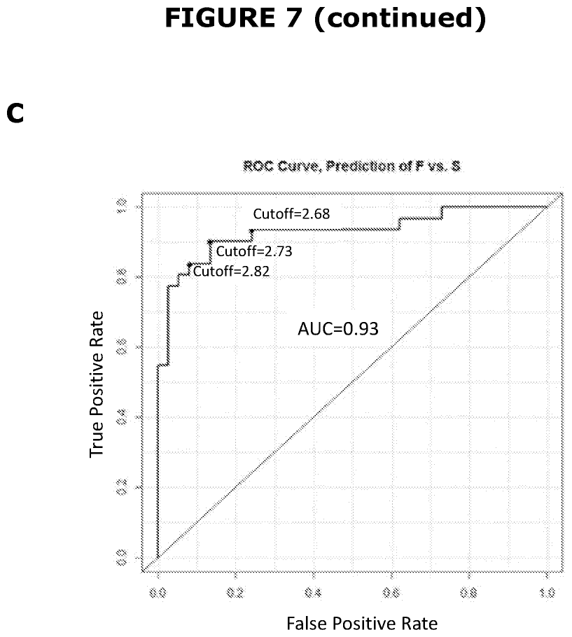

Serum sCD14 levels were correlated with increasing disease burden in patients with ALS (n=28) based on the AALS scoring system. Serum sCD14 levels were positively correlated with the patient's AALS score at the time of blood draw (p<0.0001, R=0.684) (FIG. 7A). Further, since ALS Tregs were recently shown to be dysfunctional (Henkel et al., EMBO Mol Med 2013; 5:64-79; Beers et al., JCI Insight. 2017; 2(5):e89530), the correlation of serum sCD14 levels with dysfunctional suppressive capabilities of ALS Tregs was assayed and found to be positively correlated with the patient's impaired Tregs suppressive functions (p=0.009, R=0.613) (FIG. 7B).

sCD14 Predicts Current Progression Rates

The correlations between sCD14 and disease progression rates prompted the evaluation of the sCD14 serum levels as potential indicators of the patient's current clinically assessed disease progression rate. Receiver operating characteristic (ROC) analyses (FIG. 7C) were used to evaluate the accuracy of serum levels from these patients for reflecting rapid versus slow disease progression rates at the time of serum collection (using both the progression previously described, Henkel et al., EMBO Mol Med 2013; 5:64-79). Serum sCD14 levels were an accurate indicator of disease progression rates. Using a ROC cutoff over 2.73 μg/ml of control as positive, serum sCD14 levels had a 90.9% accuracy, 88% sensitivity and 90% specificity for distinguishing rapid progressing patients from slow progressing patients.

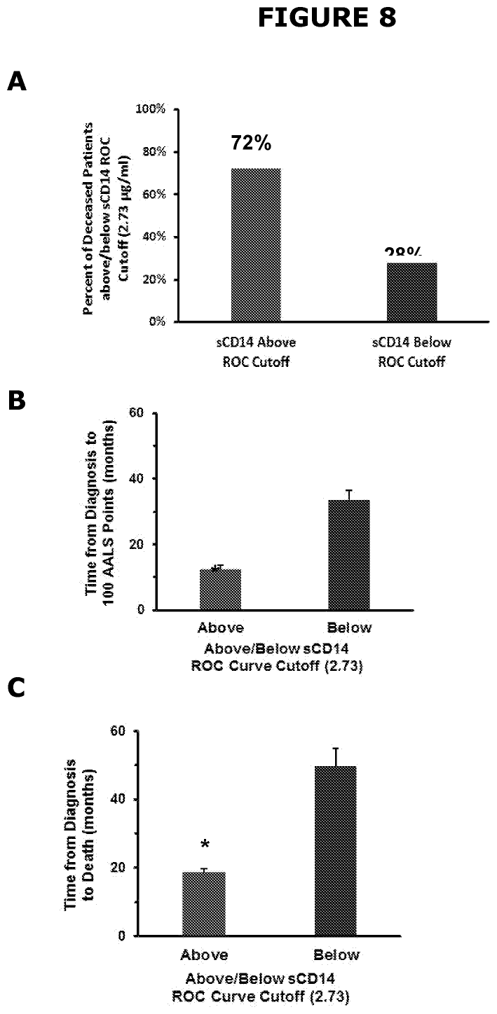

An earlier study evaluated whether a ROC score of 0.66-fold cutoff for low FOXP3 mRNA levels was predictive of a worse clinical outcome and reported that 35% of patients with FOXP3 levels below this cutoff were on a ventilator or deceased, while only 13% of patients with FOXP3 levels above the cutoff were on a ventilator or deceased. Using the ROC score cutoff of 2.73 μg/ml of serum sCD14, 72% (21/29) of patients with ALS that had sCD14 values above the cutoff were deceased, while only 28% (8/29) of patients with sCD14 values above the cutoff remained alive (FIG. 8A). A clinical scoring system for ALS disease burden revealed that patients with a ROC score above the threshold reached 100 AALS points faster than those patients with a score below threshold (FIG. 8C). As a measure of disease progression, patients with a ROC score above the cutoff lived a shorter period of time from diagnosis until death than those patients with a score below the cutoff. A Spearman's correlation coefficient analysis was used to correlate disease burden and disease progression (FIGS. 8B and 8C) with patients levels of serum sCD14. The analysis demonstrated that the greater the level of serum sCD14, the faster the patients reached 100 AALS points (FIG. 8D) and the shorter the patients' survival time from diagnosis (FIG. 8E).

### Serum Soluble LBP Concentrations

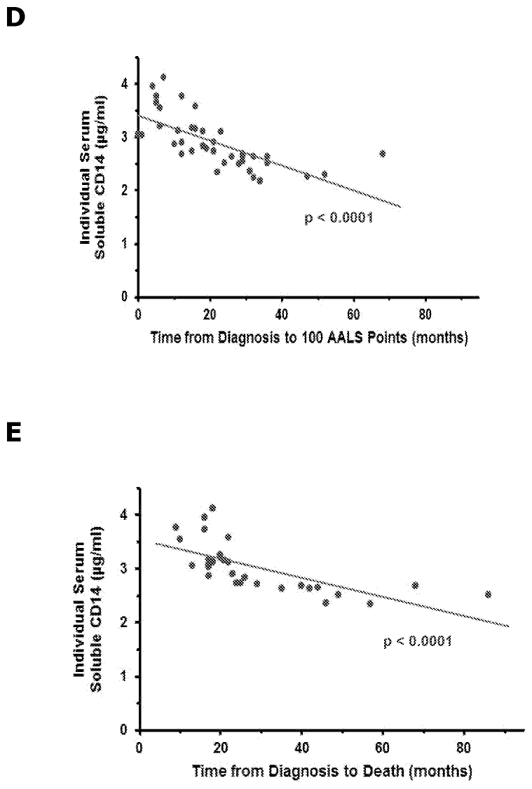

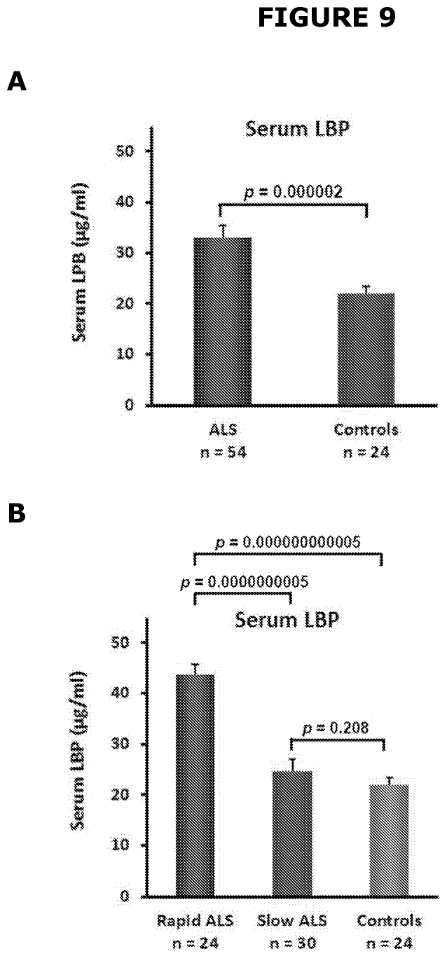

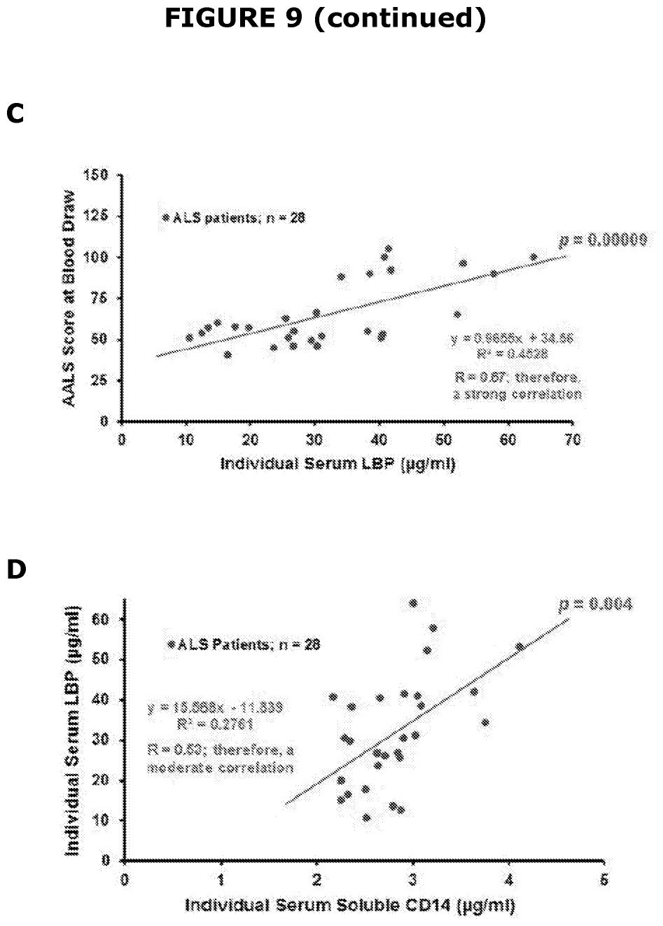

Assays were performed to detect the levels of soluble lipopolysaccharide binding protein (LBP, also referred to as sLBP) in the serum of patients and HV. LBP was increased in the serum of all patients compared with HV (p<0.0001) (FIG. 9A). When the serum samples were separated into rapid progressing and slow progressing patients, LBP was only elevated in the rapid progressing patients compared with either slow progressing patients (p<0.0001) or HV (p<0.0001) (FIG. 9B). There was no difference in serum LBP between slow progressing patients with ALS and HV (p=0.208). Interestingly, although serum LPS/endotoxin was trending up in patients with ALS, the levels did not reach significance when compared with levels in serum from HV (data not shown).

In a subset of these patients (n=28) serum LBP was positively correlated with the patient's burden of disease (p=0.001, R=0.587) (FIG. 9C); as LBP increased there was a deterioration the patient's health status as determined by an increased AALS score. Since serum sCD14 was positively correlated with disease burden, there was a positive correlation between LBP and sCD14 in these patients (p=0.001, R=0.584) (FIG. 9D); thus, as LBP increased there was a concomitant increase in sCD14.

### C Reactive Protein

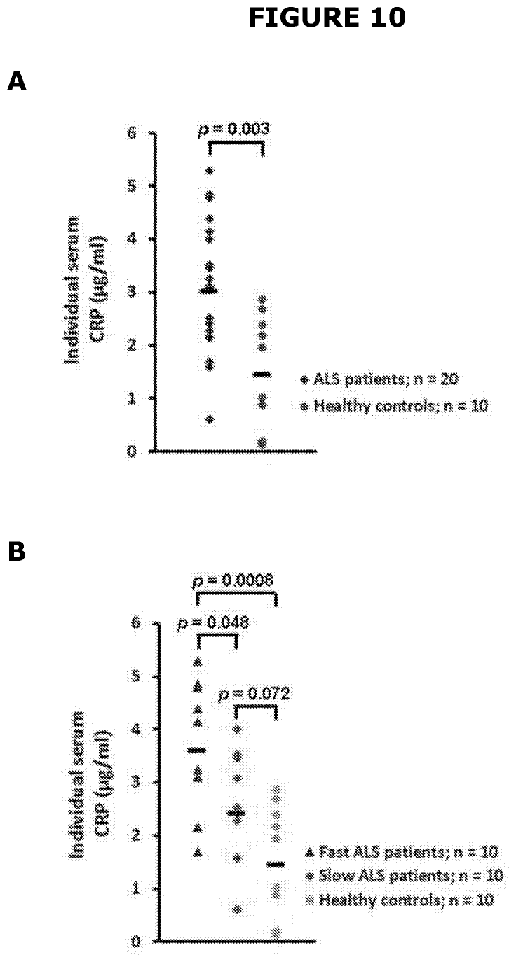

CRP was measured in the serum of patients and HV. CRP was elevated in the sera of all patients compared with HV (p=0.003) (FIG. 10A), but was only elevated in the rapid progressing patients compared with either slow progressing patients (p<0.048) or HV (p=0.0008) (FIG. 10B). There was no difference in serum CRP between slow progressing patients with ALS and HV (p=0.072).

### Serum MIF

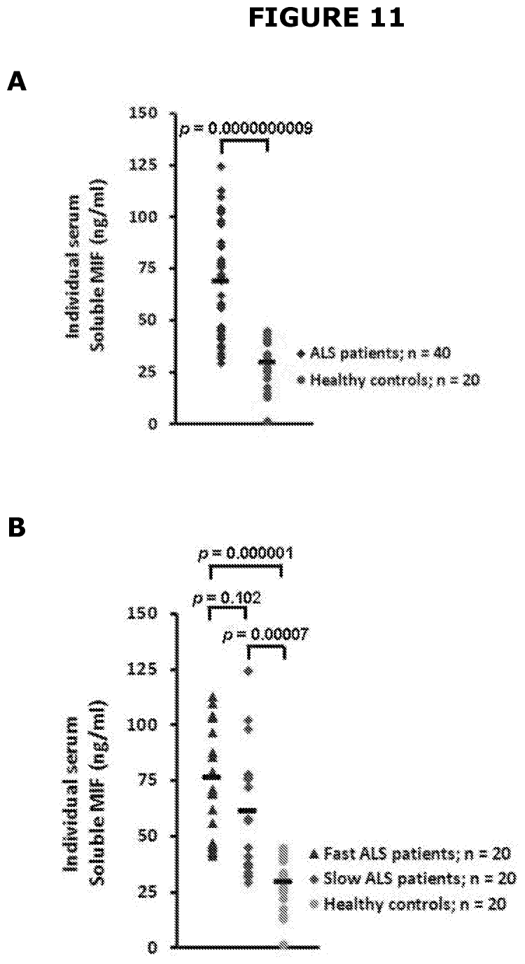

Macrophage migration inhibitory factor (MIF) was measured in the serum of patients. MIF was elevated in the serum of all patients compared with HV (p<0.0001) (FIG. 11A). When the serum samples were separated into rapid progressing and slow progressing patients, MIF was elevated in both the rapid progressing (p<0.0001) and slow progressing patients (p<0.0001) compared with HV (FIG. 11B); there was no difference between rapid progressing and slow progressing patients with ALS (p<0.102).

### Serum Soluble TNFR1 and TNFR2

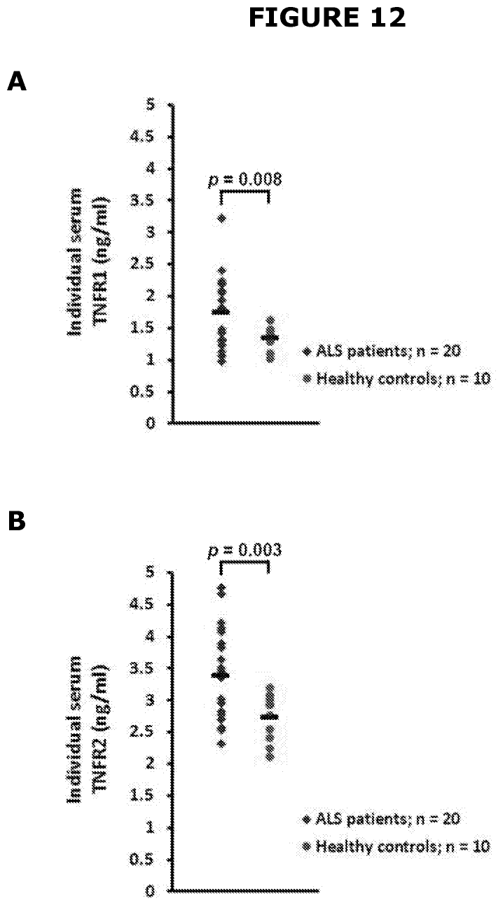

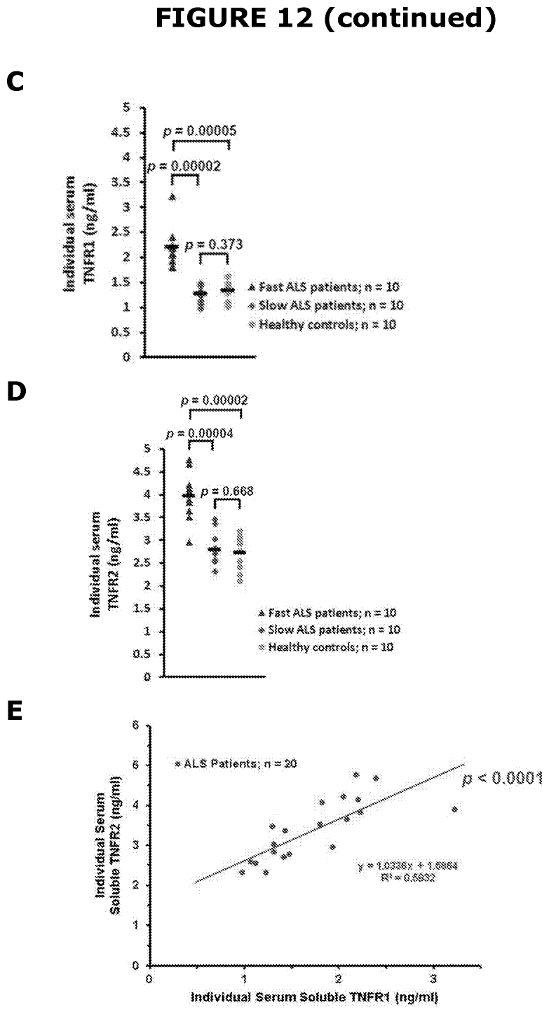

Tumor necrosis factor (TNF) is produced by monocytes/macrophages, as-well-as other cells, and is a pro-inflammatory cytokine that mediates its pleiotropic activities via two cell surface receptors, TNFRI and TNFRII, which are usually bound to the cell surface. Soluble TNFRII and TNFRII were measured in the serum of patients. Serum levels of these receptors were increased in patients compared with HV (TNFRI, p=0.008; TNFRII, p=0.003) (FIGS. 12A and B). When separated into rapid progressing and slow progressing patients, soluble TNFRI and TNFRII levels were only elevated in the rapid progressing patients compared with either slow progressing patients (TNFRI, p<0.0001; TNFRII, p<0.0001) or HV (TNFRI, p<0.0001; TNFRII, p<0.0001) (FIGS. 12C and D). There were no differences in serum soluble TNFRI and TNFRII levels between slow progressing patients and HV (TNFRI, p=0.373; TNFRII, p=0.668).

Since both soluble TNFRI and TNFRII levels were elevated in the serum of patients, and since soluble TNFRI and TNFRII levels were only elevated in rapid progressing ALS patients, a Spearman's correlation coefficient analysis was used to determine if there was an association between the serum levels of these two receptors. This analysis demonstrated that there was a positive correlation between serum soluble TNFRI and TNFRII in these patients (p<0.0001, R=0.851). Thus, as soluble TNFRI levels increased, soluble TNFRII levels also increased. However, there was not an association between TNFRI and TNFRII levels in the serum of HV (p=0.98, R=0.01).

### Discussion

Inflammation is now recognized to play a prominent role in the pathobiology of ALS (Appel et al., Trends Immunol. 2010; 31(1):7-17; Appel et al., Acta Myol. 2011; 30(1):4-8). The responses of both adaptive and innate immune systems play pivotal and interdependent roles regulating the rate of disease progression and survival. Henkel et al. (EMBO Mol Med 2013; 5:64-79) reported that Tregs have a protective role in patients with ALS, an adaptive immune response. Reduced Treg numbers and FOXP3 expression were associated with a more rapid disease progression. Low circulating levels of Tregs and FOXP3 expression were associated with increased mortality after 3.5 years, while higher levels of Tregs and FOXP3 expression were associated with lower mortality over the same period of time. Another study using deep RNA-sequencing and qRT-PCR techniques demonstrated that monocytes isolated from patients with ALS expressed a unique gene profile associated with pro-inflammatory immune responses, an innate immune response (Zhao et al., Neurol. 2017; 74(6):677-685). Nine of 10 top upregulated differentially expressed genes were involved in inflammation. The study shows that CD14^{-/low}/CD16⁺ monocytes were decreased in patients with ALS, and that cell surface expression of CD14 on monocytes is decreased. In accord with these observations, serum sCD14 levels were found to be increased in rapid progressing patients, and accurately predicted decreased survival times in this group of patients. This is the first report demonstrating that serum sCD14 levels could be used as a biomarker for disease progression. Soluble LBP, MIF, CRP, and TNFRI and TNFRII were also elevated in patients' sera. The collective profile of these factors is distinct for ALS and further distinguishes the rates of disease progression. Additionally, the collective profile is more likely to increase the specificity and sensitivity for ALS. Thus, the present study provides confirmatory as well as additional evidence for a pro-inflammatory innate immune response in patients with ALS; this response is associated with increased disease burden and rapid disease progression. This pro-inflammatory environment further contributes to the deteriorating clinical status of the patient.

CD14, a glycosylphosphatidylinositol (GPI)-anchored membrane glycoprotein, is a myeloid differentiation marker expressed predominately by monocytes/macrophages, although low levels are found on neutrophils, and along with the Toll-like receptor 4 and MD-2, acts as a co-receptor for LPS. CD16 is also expressed by monocytes/macrophages and has been identified as an Fc receptor. Based on CD14 and CD16 cell surface expression, human monocytes can be divided into three distinct subsets: classical CD14⁺/CD16⁻ monocytes, intermediate CD14⁺/CD16⁺ monocytes, and non-classical CD14^{-/low}/CD16⁺ monocytes. The most predominant of the three are the CD14⁺/CD16⁻ monocytes and they account for approximately 80% of the total monocyte population while the latter two subsets account for the remaining populations of total monocytes. This report demonstrates that non-classical monocytes, the CD14^{-/low}/CD16⁺ monocytes, were decreased in total PBMC from patients with ALS; this sub-population was decreased in both PBMC and isolated pan monocytes from rapid progressing patients. Furthermore, as shown by the MFI, the cell surface expression of CD14 is also decreased on the CD14⁺/CD16⁻ and CD14⁺/CD16⁺ monocyte sub-populations. The decreased cell surface expression of CD14 is associated with monocyte activation; CD14 is shed as sCD14 upon monocyte activation. Thus, the decreased numbers of CD14^{-/low}/CD16⁺ monocytes, and the decreased cell surface expression of mCD14 on CD14⁺/CD16⁻ and CD14⁺/CD16⁺ monocytes, provide additional evidence for a pro-inflammatory innate immune response in these patients.

The regulation of TIM-3 during immune responses suggests divergent adaptive and innate immune functions (Han et al., Front Immunol 2013; 4:449). On the one hand, TIM-3 has been identified as a negative regulator of T cell responses by inducing apoptosis; TIM-3 terminates Th1 immunity in adaptive immune responses (Hastings, et al., Eur J Immunol 2009; 39(9):2492-2501). On the other hand, studies have shown that this same TIM-3 molecule is also expressed on innate immune cells and acts synergistically with TLR signaling pathways to promote a pro-inflammatory response; TIM3 is considered a pro-inflammatory marker on monocytes and macrophages (Anderson et al., Science 2007; 318(5853):1141-1143). In contrast to the decrease in the numbers of non-classical CD14^{-/low}/CD16⁺ monocytes from patients with ALS, the frequency of CD14^{-/low}/CD16⁺/TIM-3⁺ monocytes was increased in both PBMC and isolated pan monocytes from rapid progressing patients. This is another indicator of the pro-inflammatory peripheral immune environment in these patients.

Monocyte activation-dependent shedding of mCD14 yields the vast majority of sCD14. Additionally, increased mCD14 expression or direct CD14 secretion that bypasses the GPI linkage could possibly contribute to the sCD14 variations. LPS is a potent monocyte activator that binds to CD14 and induces cellular release of sCD14, but other TLR ligands such as flagellin and CpG oligodeoxynucleotides, also induce release of sCD14 (Marcos et al., Respir Res. 2010; 11:32; Shive et al., AIDS. 201; 29(10):1263-1265). In recent years, many clinical studies focusing on sCD14 revealed that in infectious diseases, such as sepsis and HIV, sCD14 levels were elevated and associated with disease severity or worse prognosis (Shive et al., AIDS. 201; 29(10):1263-1265). Another study reported the elevation of sCD14 in urine of patients with coronary artery disease (CAD) and these levels correlated with severity of CAD (Lee et al., PLoS One. 2015; 10(2):e0117169). The present study shows that sCD14 levels were increased in the serum of patients with ALS, but when separated into rapid progressing and slow progressing patients, sCD14 levels were only elevated in the serum of rapid progressing patients. In addition, sCD14 levels were also elevated in the CSF of patients, but again, when separated into patients with different progression rates, sCD14 levels were only elevated in the CSF of rapid progressing patients. Thus, in consideration of the evidence that there are decreased numbers of CD14^{-/low}/CD16⁺ monocytes, and decreased cell surface expression of mCD14 on CD14⁺/CD16⁻ and CD14⁺/CD16⁺ monocytes, the elevated levels of serum sCD14 are most likely to be due to the activation-dependent cleavage and shedding of mCD14 from monocytes. Since it is known that parenchymal microglia also express mCD14, a possible source of the elevated CSF sCD14 levels may be due to the shedding of mCD14 from activated microglia, in addition to the shedding mCD14 from activated monocytes/macrophages in this compartment. Therefore, these data again support the concept of a pro-inflammatory monocyte response in patients with ALS, and especially in patients with rapid progressing disease.

As previously mentioned, earlier studies have shown that monocytes produce sCD14 by proteolytic cleavage of mCD14. However, it is also known that human monocytes produce sCD14 by a protease-independent mechanism; sCD14 is released from intracellular pools of CD14. To determine if CD14 is actively produced in PBMC, and thus increasing the intracellular pool of CD14 and its possible release from these cells as sCD14, PBMC were assayed by qRT-PCR for CD14 mRNA levels. The CD14 mRNA was reduced in PBMC from patients, but when separated into rapid progressing and slow progressing patients, CD14 mRNA was only reduced in the rapid progressing patients. Therefore, while sCD14 was increased in sera of rapid progressing patients, CD14 mRNA from PBMC of these same patients was decreased, suggesting the increased serum levels of sCD14 was due to cleavage from monocyte surface and not due to the release of sCD14 from intracellular pools of CD14. These data, when considered with the previous evidence, again suggest that the elevated levels of serum sCD14 are mediated by the activation of monocytes.

This is the first report showing the elevation of serum sCD14 in a neurodegenerative disease. As such, serum sCD14 levels were also evaluated in other neurological diseases such as patients with dementia (Alzheimer's disease and frontal temporal dementia) or CIDP, another neurodegenerative disease and an auto-immune neurological disease, respectively. In has recently been demonstrated that increased inflammation is associated with the worsening of AD symptoms. Triggering Receptor Expressed on Myeloid Cells 2 (TREM2) is a V-type immunoglobulin (Ig) domain-containing transmembrane protein that is expressed on mononuclear phagocytes such as microglia, osteoclasts, and macrophages (Colonna and Wang, Nat Rev Neurosci. 2016 April; 17(4):201-7). Several recent studies have described TREM2-dependent phenotypes in mouse models of amyloidosis that suggests that TREM2 plays an important role in regulating the response of innate immunity in AD pathology. Furthermore, elevations in the CSF levels of soluble TREM2 fragment that implicateTREM2 in inflammatory pathways that are coincident with neuronal damage and the onset of clinical dementia. CIDP is caused by inflammation that destroys myelin of nerves, and typically involves both motor and sensory nerve dysfunction. CIPD is sometimes thought of as the chronic form of the acute inflammatory demyelinating polyneuropathy Guillain Barré syndrome. The main cellular components of this peripheral nerve inflammatory response are epineurial and endoneurial T lymphocytes and macrophages with macrophage-mediated myelin stripping. Thus, both of these neurological diseases involve monocytes/macrophages/microglia with the possibility of mCD14 shedding upon activation of these cells. These additional patients were evaluated for serum sCD14 levels to possibly distinguish ALS from these other neurological diseases. No differences found in the serum levels of sCD14 from patients with dementia or CIPD. Furthermore, when patients with dementia were separated into patients with Mild AD, AD, or FTD, no differences in serum sCD14 were found among the three different groups of patients. Thus, elevated levels of serum sCD14 are distinct for ALS and distinguish ALS from two other neurological diseases. These data further suggest that the innate peripheral immune response in ALS is associated with an enhanced monocyte activation, compared with other neurological diseases, with subsequent shedding of mCD14.

Previous studies have demonstrated in both patients with ALS and animal models of ALS, as disease progresses and disease burden escalates, there is a concomitant increase in innate immune pro-inflammatory responses (Beers et al., Proc Natl Acad Sci USA. 2008; 105(40):15558-63, Beers et al. Brain 2011; 134: 1293-1314, Beers et al. Brain Behav Immun. 2011; 25(5):1025-35; Henkel et al., EMBO Mol Med 2013; 5:64-79; Beers et al., JCI Insight. 2017; 2(5):e89530). It has been well known for several decades that there is pro-inflammatory innate microglial response in the CNS in both patients and animals with ALS. However, the role of innate immunity in the peripheral nervous system (PNS) has not been well defined. An earlier study characterized the activation of CD169/CD68/Iba1⁺ macrophages throughout the PNS in mutant transgenic ALS mice (Chiu et al., Proc Natl Acad Sci USA. 2009; 106(49):20960-5). Macrophage activation occurred pre-symptomatically and expanded from focal arrays within nerve bundles to a tissue-wide distribution following symptom onset. This study revealed a progressive innate immune response in peripheral nerves that is separate and distinct from spinal cord immune activation in these mice. More recent studies have demonstrated the presence of monocytes/macrophages surrounding degenerating peripheral nerve fibers which is an early event that proceeds the onset of clinical signs of motor weakness in ALS transgenic mice (Lincecum et al., Nat Genet. 2010; 42(5):392-9, Kano et al., Neurology. 2012; 78(11):833-5). Furthermore, CD68⁺ macrophages were observed within peripheral nerves of presymptomatic ALS mice and these inflammatory cells infiltrated only after demonstrable denervation. This later study also demonstrated increased expression of CCL2 concomitant with the appearance of CD68⁺ macrophages suggesting CCL2, possibly secreted by Schwann cells, may regulate infiltration of these cells in these peripheral nerves. More essential to the present study, Murdock et al. (JAMA Neurol. 2017; 74(12):1446-1454) recently assessed peripheral inflammatory responses in rapid progressing patients with ALS and reported increased numbers of total leukocytes, neutrophils, CD16⁺ and CD16⁻ monocytes, and natural killer cells. An acute, transient increase in CD11b⁺ myeloid cells was also observed, and these early changes in immune cell numbers were positively correlated with rates of disease progression. Although the current study did not count absolute numbers of monocytes, it does demonstrate that CD14^{-/low}/CD16⁺/TIM-3⁺ monocytes were increased in both PBMC and isolated pan monocytes from rapid progressing patients, and that this increased percentage of CD14^{-/low}/CD16⁺/TIM-3⁺ monocytes was positively correlated with rates of disease progression; the greater the percentage of CD14^{-/low}/CD16⁺/TIM-3⁺ monocytes, the more rapid the disease progression. In addition, the current study also shows a positive correlation between the percentage of CD14^{-/low}/CD16⁺/TIM-3⁺ monocytes and the AALS score; the greater the percentage of CD14^{-/low}/CD16⁺/TIM-3⁺ monocytes, the greater the escalation of disease burden. Therefore, in addition to the cross-talk between the CNS and the immune system in this compartment, cross-talk also occurs between the PNS and innate immune systems throughout the PNS during ALS disease progression (Henkel et al., J Neuroimmune Pharmacol. 2009; 4(4):389-98; Appel et al., Acta Myol. 2011; 30(1):4-8; Zhao et al., Neurol. 2017; 74(6):677-685; Hooten et al., Neurotherapeutics. 2015; 12(2):364-75).

In the ALS transgenic mouse model, the lack of Tregs exacerbated the inflammatory process, leading to accelerated motoneuron death, a more rapid disease progression, and shortened survival (Beers et al., Brain 2011; 134: 1293-1314). In patients with ALS, Treg numbers and FOXP3 mRNA expression were decreased in rapid progressing patients, and ALS Tregs were less effective in suppressing Tresp proliferation; although both rapid and slow progressing patients had dysfunctional Tregs, the greater the clinically assessed disease burden or the more rapid progressing the patient, the greater the Tregs dysfunction (Henkel et al., EMBO Mol Med 2013; 5:64-79; Beers et al., JCI Insight. 2017; 2(5):e89530). Furthermore, and as already mentioned, an earlier study reported that a ROC score cutoff of 0.66-fold cutoff accurately predicted that low FOXP3 mRNA levels was associated with a worse clinical outcome and that 35% of patients with FOXP3 levels below this cutoff were on a ventilator or deceased, while only 13% of patients with FOXP3 levels above this cutoff were on a ventilator or deceased. Using an identical ROC analyses for distinguishing patients with either rapid or slow disease progression rates at the time of serum collection, serum sCD14 levels were an accurate indicator of disease progression rates; using a ROC cutoff of 2.73 μg/ml of control as positive, serum sCD14 levels accurately distinguished patients with rapid progressing disease from slow progressing patients at a 90.9% accuracy, 88% sensitivity and 90% specificity. Furthermore, and in contrast with the earlier report (Henkel et al., EMBO Mol Med 2013; 5:64-79), these ROC analyses determined that 72% of patients that had sCD14 values of above the cutoff were deceased, while only 28% of patients with sCD14 values above the cutoff remained alive at the end of this 4 year study. In addition, 70% of patients were deceased with serum sCD14 values above the ROC cutoff while only 30% of patients were deceased with sCD14 values below the cutoff. These data demonstrated that the greater amount of sCD14 in the serum, the faster the patients reached an AALS score of 100 points and the faster the progression from diagnosis to death. Thus, these data not only suggest that the increased percentage of CD14^{-/low}/CD16⁺/TIM-3⁺ monocytes are associated with increased rates of disease progression and augmented disease burden, but also that increased serum sCD14 levels are also a direct biomarker of increased rates of disease progression and augmented disease burden. Again, this suggests that increased serum sCD14levels are indicative of monocyte activated mCD14 shedding, a direct measure of an enhanced innate immune response.

The precise role of CD14 in physiological and pathological situations is not well defined (Bas et al., J Immunol. 2004; 172(7):4470-9). The serum sCD14 is due to the cleavage and shedding of mCD14 from monocytes. At the monocyte surface, mCD14 and LBP, an acute-phase protein (APP), interact in a stepwise manner with LPS to form a high affinity tri-molecular complex that allows monocytes to detect the presence of LPS. This complex in turn interacts with Toll-like Receptor 4 (TLR4) and MD-2 for downstream signaling (Zhou et al., 2016). In contrast, two opposite functions have been described for sCD14. It can either reduce endotoxin-induced activities by competing with mCD14 for LPS binding or it can mediate the LPS-induced activation of non-CD14-expressing endothelial, epithelial, and smooth-muscle cells. sCD14 also could be an APP because apart from protease-mediated shedding from monocytes, sCD14 is produced by hepatocytes, which represent the major source of APPs (Pan et al., J Biol Chem. 2000; 275(46):36430-5; Bas et al., J Immunol. 2004; 172(7):4470-9). Interestingly, hepatocytes are also a major source of LBP. Therefore, LBP levels were also evaluated in the serum of patients with ALS. LBP was increased in the serum of all patients, but when the samples were separated into rapid progressing and slow progressing patients, LBP was only elevated in the serum of rapid progressing patients, as was shown for sCD14. Furthermore, as was again shown for sCD14, serum LBP was positively correlated with the patient's burden of disease. More intriguing, there was a positive correlation between LBP and sCD14 in these patients; as sCD14 increased there was a concomitant increase in LBP. As a possible reason for why free endotoxin/LPS was not increased in the serum of patient with ALS, in contrast to a previous report (Zhang et al., 2009), is that LPS is bound in the sCD14/LBP complex which may then be interacting TLR4 on the surface of cells not expressing mCD14, such as endothelial or epithelial cells, and thus exacerbating a pro-inflammatory response.

CRP is a classical APP regulated by pro-inflammatory cytokines and secreted by hepatocytes. CRP has prognostic value for several types of tumors, cardiovascular diseases, and rheumatic diseases. An earlier report determined that there was a positive correlation between levels of wide range CRP and clinically assessed disabilities in patients with ALS (Keizman et al., Acta Neurol Scand. 2009; 119(6):383-9). A recent study with a larger cohort of patients with ALS also found that serum CRP was elevated in these patients and patients with high levels of CRP progress more rapidly than do those with lower CRP levels (Lunetta et al., JAMA Neurol. 2017; 74(6):660-667). Since sCD14 and LBP, both APPs, were elevated, and since CRP was elevated in previous studies, CRP was evaluated in this study. CRP was elevated in the serum of all patients, but when the patients were separated into rapid and slow progressing patients, CRP was only elevated in the rapid progressing patients. Thus, these data confirm the two earlier reports and possibly suggests that the liver is not a well-recognized participant and modulator of the pro-inflammatory response in this disease.

The pro-inflammatory cytokine MIF is fundamental to innate and adaptive immune responses; MIF is a key mediator of the systemic inflammatory response. MIF has been characterized as a physiologic counter-regulator of the anti-inflammatory activities of glucocorticoids. In response to a variety of stimuli, MIF is released from preformed intracellular pools. MIF promotes the production of a number of pro-inflammatory moieties and is required for normal leukocyte influx into inflamed tissues. Elevated serum levels of MIF are detected in many infectious and inflammatory diseases, and as has been shown for sCD14, MIF levels are elevated in sepsis. MIF was found to be elevated in the serum of all patients with ALS, but unlike sCD14, LBP, and CRP, MIF was elevated in both rapid and slow progressing patients. Thus, these data suggest that MIF could possibly be an early indicator of the pro-inflammatory response occurring in ALS, and MIF levels increase as rates of progression increase.

Tumor necrosis factor-α (TNF-α) is produced by monocytes/macrophages, as-well-as other cells, and is a pro-inflammatory cytokine that mediates its pleiotropic activities via two cell surface receptors, TNFRI and TNFRII. Both receptors are ubiquitously expressed and display structurally similar extracellular domains but signal through distinct intracellular regions, with TNFRI containing a death domain that is not present in TNFRII. The two TNFRs are released as soluble proteins by proteolytic cleavage of their extracellular domains, in exosomes, or via alternative splicing of mRNA transcripts which leads to a loss of the transmembrane and cytoplasmic domains. Soluble TNFRs can act as TNF antagonists and can inhibit TNF-mediated pro-inflammatory effects. However, the soluble receptors can stabilize and preserve circulating soluble TNF and thus function as TNF agonists. Therefore, soluble TNFRs may play a role as modulators of the biological activity of TNF-α. TNFRI predominantly promotes inflammatory signaling pathways, whereas TNFRII mediates immune modulatory functions and promotes tissue homeostasis and regeneration. In multiple sclerosis (MS), there is a TNFRI polymorphism that is associated with susceptibility (Gregory et al., Nature 2012, 488, 508-511). The function of this polymorphism is to produce a new TNFRI variant that is secreted and binds TNF-α. In MS patients, early in the disease, excess TNF receptor shedding has been found in the blood suggesting lower bioavailability of TNF. Several studies have reported that TNFRII is preferentially expressed on a maximally suppressive subset of human and mouse Tregs, and activation of TNFRII is important for the proliferation and function of Tregs (Chen et al., J. Immunol. 2007, 179, 154-161; Chen et al. J. Immunol. 2008, 180, 6467-6471, Chen et al., Curr. Dir. Autoimmun. 2010, 11, 119-134, Chen et al. J. Immunol. 2013, 190, 1076-1084; Heinrich et al., Antibodies 2015; 4:34-47). In the current study, both TNFRs were elevated in the sera of all patients with ALS, but as has been found throughout this study, when the patients were separated into rapid and slow progressing patients, the receptors were only elevated in the rapid progressing patients. Poloni et al. (Neurosci Lett. 2000; 287(3):211-4) reported that TNF-α and the soluble receptors levels were elevated in serum of patients with ALS. Interestingly, the biologically active form of TNF-α, corresponding to the unbound trimeric molecule, was similar between patients and HV. A later study also found TNF-α levels elevated in the serum of a smaller cohort of patients with ALS (Babu et al., Neurochem Res 2008; 33: 1145-1149). With respect to these two earlier studies and the current study, these data suggest a duel immune response in patients. First, because of the known inflammatory responses associated with pathobiology of ALS, the two receptors are shed to act as TNF antagonists and inhibit the pro-inflammatory effects TNF-α. In consideration of these data and the recent report that Tregs isolated from rapid progressing patients are highly dysfunctional, and to a lesser extent Tregs from slow progressing patients, shedding of TNFRII from the surface of Tregs, thus blocking TNF-α signaling through TNFRII, may inhibit the suppressive functions of these cells (Beers et al., JCI Insight. 2017; 2(5):e89530). Second, the elevation of serum TNFRs may bind TNF-α, and act as a reservoir for future TNF-α release and its subsequent pro-inflammatory responses.

It is well documented that there is a microglia-directed pro-inflammatory response in CNS tissues of patients with ALS (Turner et al., Neurobiol Dis 2004; 5: 601-609; Appel et al., Acta Myol. 2011; 30(1):4-8; Corcia et al., PLoS One 2012; 7:e52941; Brites and Vaz, Front Cell Neurosci. 2014; 8: 117). Accordingly, the data presented in this report support the concept of an ongoing monocyte directed pro-inflammatory environment that exists in the peripheral circulation of these patients, especially those patients with rapid progressing disease (Zhao et al., Neurol. 2017; 74(6):677-685). The decreased numbers of CD14^{-/low}/CD16⁺ monocytes, the decreased cell surface expression of mCD14 on CD14⁺/CD16⁻ and CD14⁺/CD16⁺ monocytes, and the increased percentage of CD14^{-/low}/CD16⁺/TIM-3⁺ monocytes in these patients are in accord with this concept. More importantly, serum sCD14 levels were found to be increased in rapid progressing patients, and accurately predicted decreased survival times. Including the additional evidence that soluble LBP, MIF, CRP, and TNFRI and TNFRII were also elevated in patients' sera, these factors and sCD14 constitute a collective immune biomarker profile that are distinct for ALS, and are likely to increase the biomarker specificity and sensitivity for this disease.

### Example 2

A second study using a larger cohort of patients was conducted to confirm the findings above in relation of the ability of sCD14 and LBP to act as biomarkers for ALS progression rates. In this study, 100 ALS patients and 60 healthy volunteers (HV) were assessed essentially as described above to determine levels of sCD14 and LBP in serum.

Progression rates of the patients with ALS were calculated from the Appel ALS (AALS) scoring system (Haverkamp et al., 1995 Brain 118:707-719; Voustianiouk et al., 2008 Muscle Nerve. 37(5):668-72). The AALS scores ranged from 30 points (HV) to 164 points (severe ALS). To determine progression rates, the patient's AALS score at their last visit to the hospital was subtracted from their score at their initial visit to the hospital, and then that number was divided by the number of months between the two visits. A score of equal to or greater than 1.5 AALS pts/month was representative of a rapid (or fast) progressing patient, and a score less than 1.5 AALS pts/month was representative of a slow progressing patient.

Serum sCD14

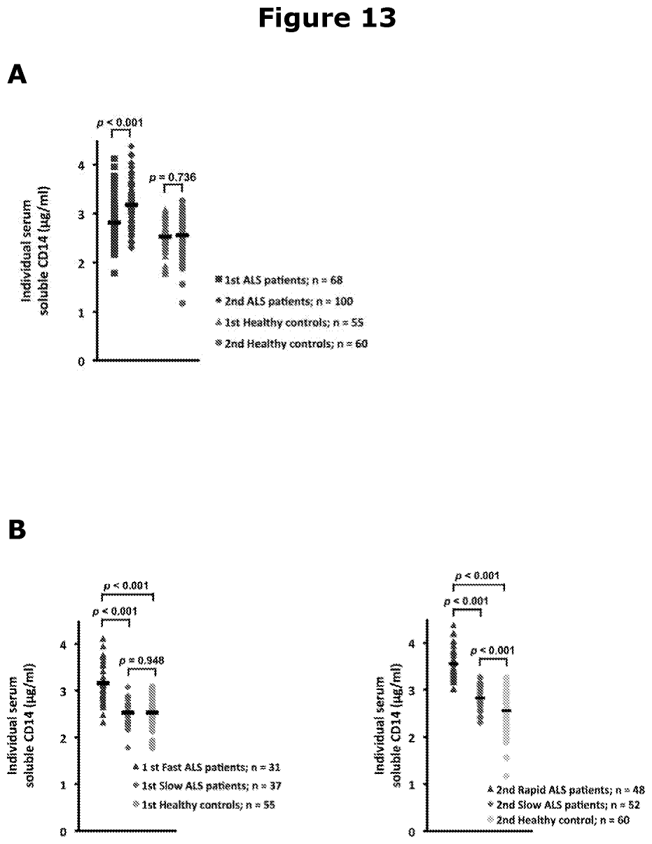

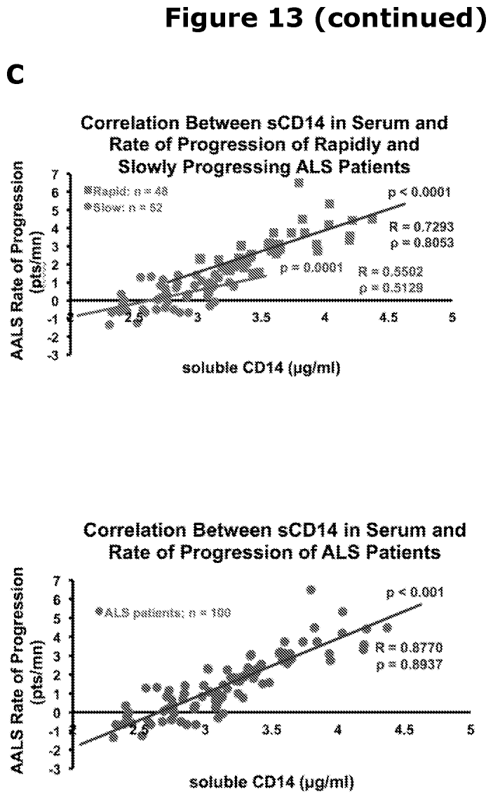

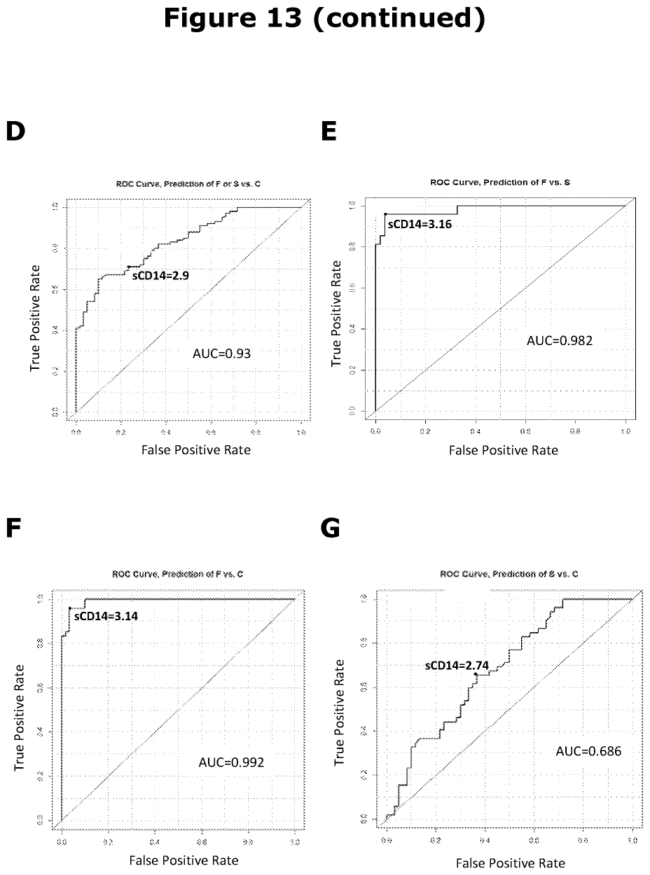

An analysis of the levels of sCD14 in the serum of patients with ALS in this cohort essentially confirmed the finding in the first study (Example 1) that level of sCD14 in ALS patients was significantly increased compared to HV (FIG. 13A). Of interest, while this second study also identified a clear difference between sCD14 levels in rapid progressing versus slow progressing patients, as was shown in the first study, it also demonstrated a statistically significant increase in sCD14 levels in slow progressing patients compared to HV (FIG. 13B). Overall, there was a clear correlation between sCD14 levels in serum and the rate of progression of disease in ALS patients (FIG. 13C).

Receiver Operating Characteristic (ROC) curve analyses were used to evaluate the accuracy of sCD14 serum levels from this cohort of patients for reflecting rapid versus slow disease progression rates at the time of serum collection (FIG. 13D-G). As shown in FIG. 13E, serum sCD14 levels were a clear indicator of disease progression rates, able to very accurately distinguish rapid versus slow progressing patients (ROC cutoff of 3.16 μg/ml; AUC=0.982; sensitivity: 0.942; specificity: 0.958). To a lesser extent, serum sCD14 levels could also distinguish slow progressing patients from HV (FIG. 13G), (ROC cutoff of 2.74 μg/ml; AUC=0.686; sensitivity: 0.633; specificity: 0.654).

### Serum LBP

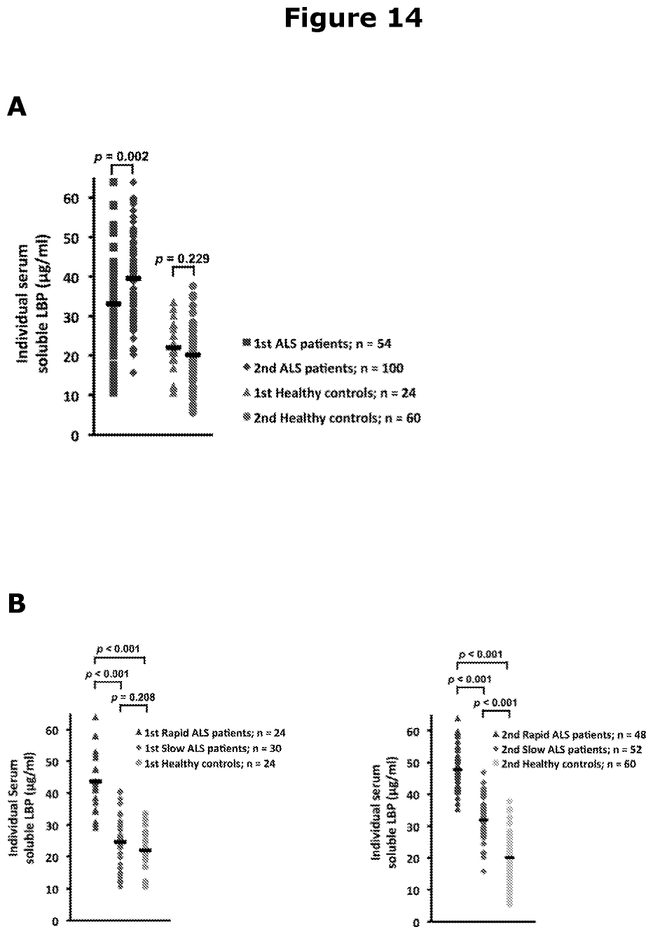

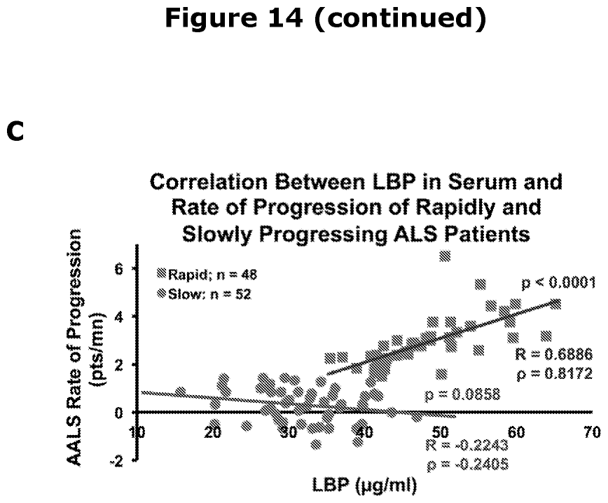

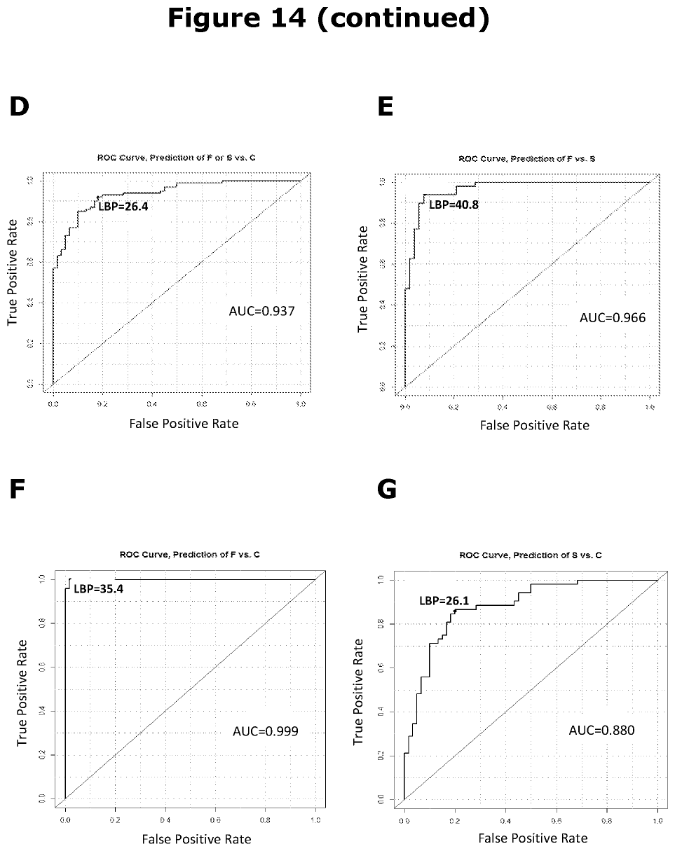

An analysis of the levels of LBP in the serum of patients with ALS in this cohort also essentially confirmed the finding in the previous study that level of LBP in ALS patients was significantly increased compared to HV (FIG. 14A). As with sCD14, while this second study also identified a difference between LBP levels in rapid progressing patients compared to slow progressing patients (as was shown in the first study), it also demonstrated a statistically significant increase in LBP levels in slow progressing patients over HV (FIG. 14B). Overall, the clear correlation between LBP levels in serum and the rate of progression of disease in ALS patients that was observed in the first study was also observed in this second study (FIG. 14C).

Using ROC analyses to evaluate the accuracy of serum LBP levels from this cohort of patients for reflecting rapid versus slow disease progression rates, it was clear that, like sCD14, LBP could also be used as a biomarker to accurately distinguish the various progression rates (FIG. 14D-G). As shown in FIG. 14E, serum LBP levels were a good indicator of disease progression rates, able to very accurately distinguish rapid compared to slow progressing patients (ROC cutoff of 40.8 μg/ml; AUC=0.966; sensitivity: 0.923; specificity: 0.938). In this study, serum LBP levels could also distinguish slow progressing patients from HV (FIG. 13G) (ROC cutoff of 26.1 μg/ml; AUC=0.880; sensitivity: 0.783; specificity: 0.865).

Serum sCD14 and LBP

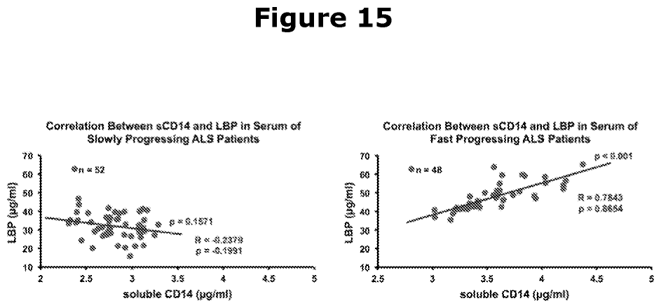

As observed in the first study, there was a clear correlation between levels of sCD14 and LBP in patients with rapid progressing ALS, a correlation that was not seen in patients with slow progressing ALS (FIG. 15A).

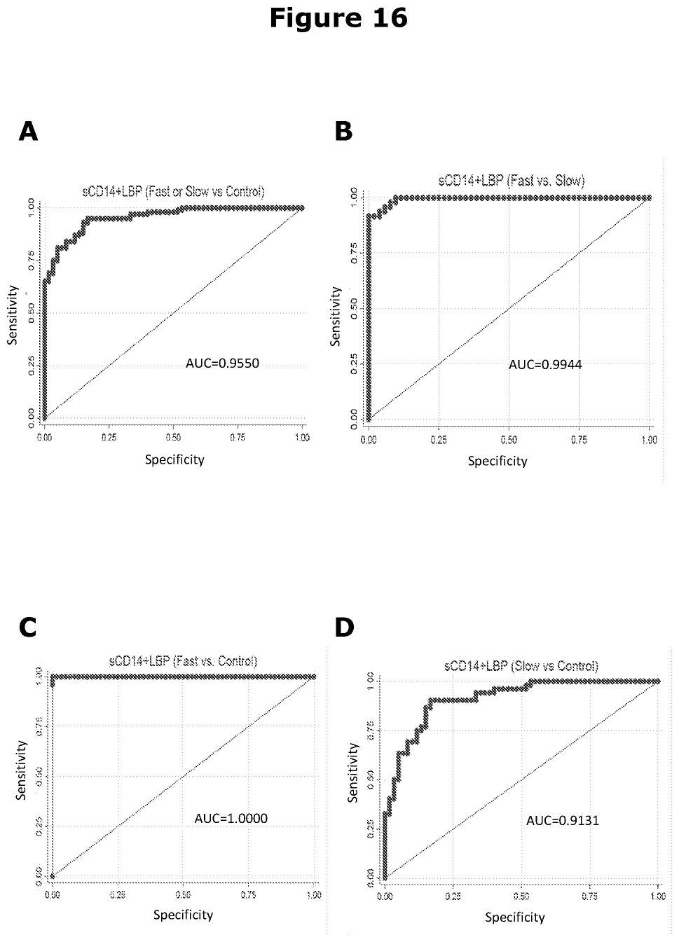

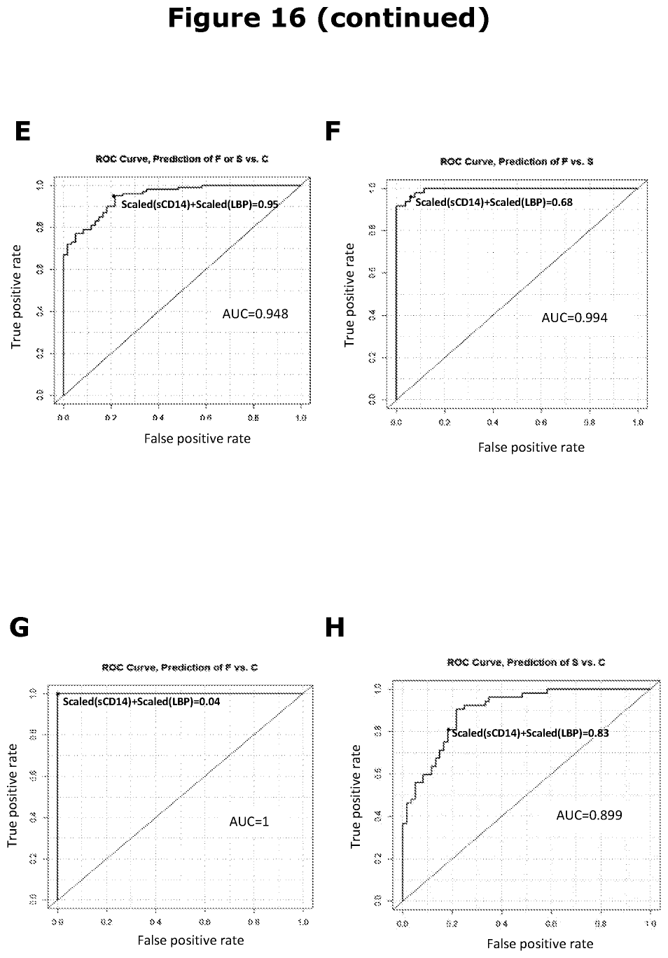

To determine whether the combination of serum sCD14 and serum LBP levels could be used to more effectively distinguish between slow and rapid progressing ALS compared to either biomarker alone, ROC analyses were performed (FIG. 16). It was demonstrated that the combination of serum sCD14 and serum LBP levels was particularly effective at predicting whether a patient with ALS had slow or rapid progressing disease. This was demonstrated using actual serum levels of sCD14 and LBP in the analyses (FIG. 16B; AUC=0.9944), and used scaled levels of serum sCD14 plus LBP (FIG. 16F; AUC=0.994). In the latter analyses, since levels of sCD14 and LBP are a degree of magnitude apart, the levels of each protein were first standardized and then added to create a new measurement− scaled(sCD14)+ scaled(LBP).

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the disclosure without limiting the disclosure to any one embodiment or specific collection of features. Those of skill in the art will therefore appreciate that, in light of the instant disclosure, various modifications and changes can be made in the particular embodiments exemplified without departing from the scope of the present disclosure. All such modifications and changes are intended to be included within the scope of the appended claims.

What is claimed is:

1. A method for determining whether a subject with Amyotrophic Lateral Sclerosis (ALS) is likely to have rapid or slow progressing ALS, the method comprising:
(a) determining the level of a biomarker in a biological sample obtained from a subject with ALS, wherein the biomarker is soluble CD14 (sCD14) or lipopolysaccharide binding protein (LBP); and
(b) determining whether the subject is likely to have rapid or slow progressing ALS based on the level of the biomarker in the biological sample relative to a suitable reference level.

2. The method of claim 1, wherein the reference level is representative of a healthy subject and/or a subject known to have slow progressing ALS, and wherein an increase in the level of the biomarker relative to the reference level indicates that the subject is likely to have rapid progressing ALS.

3. The method of claim 1, wherein the reference level is representative of a subject known to have rapid progressing ALS, and wherein a similar level of the biomarker relative to the reference level indicates that the subject is likely to have rapid progressing ALS.

4. The method of claim 1, wherein the reference level is representative of a healthy subject and/or a subject known to have slow progressing ALS, and wherein a similar level of the biomarker relative to the reference level indicates that the subject is likely to have slow progressing ALS.

5. The method of claim 1, wherein the reference level is representative of a subject known to have rapid progressing ALS, and a decrease in the level of the biomarker relative to the reference level indicates that the subject is likely to have slow progressing ALS.

6. The method of claim 1, wherein the reference level is a threshold level above which the subject is likely to have rapid progressing ALS, and below which the subject is likely to have slow progressing ALS.

7. A method for assessing the rate of progression of ALS in a subject, the method comprising:
(a) determining the level of a biomarker in a biological sample obtained from a subject with ALS, wherein the biomarker is soluble CD14 (sCD14) or lipopolysaccharide binding protein (LBP); and
(b) determining the rate of progression of ALS in the subject based on the level of the biomarker in the biological sample.

8. The method of any one of claims 1 to 7, wherein the method comprises determining the level of sCD14 and LBP.

9. The method of any one of claims 1 to 8, wherein the biological sample is selected from the group consisting of blood, plasma, serum, urine and cerebrospinal fluid (CSF).

10. The method of any one of claims 1 to 9, further comprising measuring the level of at least one other biomarker in the biological sample.

11. The method of claim 10 wherein the at least one other biomarker is selected from the group consisting of LBP, CRP, MIF, sTNFRI and/or sTNFRII.

12. The method of any one of claims 1 to 11, further comprising obtaining the biological sample prior to measuring the level of the biomarker.

13. The method of any one of claims 1 to 12, further comprising exposing the subject to a treatment regimen for treating ALS.

14. A kit for determining the level of a biomarker in a subject with ALS, wherein the kit comprises an antigen-binding molecule specific for the biomarker which allows for measuring the level of the biomarker in a biological sample, wherein the biomarker is sCD14 or LBP.

15. The kit of claim 14, comprising an antigen-binding molecule specific for sCD14 and an antigen-binding molecule specific for LBP, which allow for measuring the level of sCD14 and LBP in a biological sample.

16. The kit of claim 14 or 15, further comprising an antigen-binding molecule specific for at least one other biomarker selected from among CRP, MIF, sTNFRI, sTNFRII, NFL, pNfH, p75NTR^{ECD}, miR-206, miR-143-3p, and miR-374b-5p.

17. A solid support, comprising an antigen-binding molecule specific for a biomarker, wherein the biomarker is sCD14 or LBP.

18. The solid support of claim 17, comprising an antigen-binding molecule specific for sCD14 and an antigen-binding molecule specific for LBP.

19. The solid support of claim 17 or 18, further comprising an antigen-binding molecule specific for at least one other biomarker selected from among CRP, MIF, sTNFRI, sTNFRII, NFL, pNfH, p75NTR^{ECD}, miR-206, miR-143-3p, and/or miR-374b-5p.

20. The solid support of any one of claims 17 to 19, wherein the solid support is selected from among a multiwell plate, a slide, a chip or a plurality of beads.

21. A method of stratifying a subject to a treatment for ALS, the method comprising:
(a) determining whether or not a subject is likely to have rapid or slow progressing ALS, or assessing the rate of progression of ALS in a subject, in accordance with the method of any one of claims 1 to 12; and
(b) determining an optimized treatment regime suitable for the subject based on whether the subject is likely to have rapid or slow progressing ALS, or based on the rate of progression of ALS in the subject.

22. The method of claim 21, further comprising exposing the subject to the optimized treatment regimen.

23. A method for treating a subject that is likely to have rapid progressing ALS, the method comprising:
(a) selecting a subject that is likely to have rapid progressing ALS on the basis the level of a biomarker in a biological sample of the subject, wherein the biomarker is sCD14 or LBP; and
(b) exposing the subject to a treatment regimen optimized for treating rapid progressing ALS.

24. The method of claim 23, wherein (a) comprises selecting a subject that is likely to have rapid progressing ALS on the basis of the levels of sCD14 and LBP in the biological sample of the subject.

25. The method of claim 23 or 24, further comprising, prior to selecting the subject, determining whether the subject is likely to have rapid progressing ALS in accordance with the method of any one of claims 1-12.

26. The method of any one of claims 23 to 25, wherein the treatment regimen comprises the administration of an anti-neurodegenerative agent.

27. The method of claim 26, wherein the anti-neurodegenerative agent is selected from among riluzole, edaravone, a CD14 antagonist, GM604, masitinib, a complement pathway inhibitor, and an agent that blocks the interaction between CD40 and CD40 ligand.

28. The method of claim 27, wherein the CD14 antagonist is a CD14 antagonist antibody.

29. The method of claim 28, wherein the CD14 antagonist antibody is selected from:
(1) an antibody comprising:
a VL domain that comprises, consists or consists essentially of the sequence:
[SEQ ID NO: 1]
QSPASLAVSLGQRATISCRASESVDSFGNSFMHWYQQKAGQPPKSSIYRA
ANLESGIPARFSGSGSRTDFTLTINPVEADDVATYFCQQSYEDPWTFGGG
TKLGNQ (3C10 VL);
and
a VH domain that comprises, consists or consists essentially of the sequence: LVKPGGSLKLSCVASGFTFSSYAMSWVRQTPEKRLEWVASISSGGTTYYPDNVKGRFTISRDNA RNILYLQMSSLRSEDTAMYYCARGYYDYHYWGQGTTLTVSS [SEQ ID NO: 2] (3C10 VH);
(2) an antibody comprising:
a VL domain that comprises, consists or consists essentially of the sequence:
[SEQ ID NO: 3]
QSPASLAVSLGQRATISCRASESVDSYVNSFLHWYQQKPGQPPKLLIYRA
SNLQSGIPARFSGSGSRTDFTLTINPVEADDVATYCCQQSNEDPTTFGGG
TKLEIK (28C5 VL);
and
a VH domain that comprises, consists or consists essentially of the sequence:
[SEQ ID NO: 4]
LQQSGPGLVKPSQSLSLTCTVTGYSITSDSAWNWIRQFPGNRLEWMGYIS
YSGSTSYNPSLKSRISITRDTSKNQFFLQLNSVTTEDTATYYCVRGLRFA
YWGQGTLVTVSA (28C5 VH);
and
(3) an antibody comprising:
a VL domain that comprises, consists or consists essentially of the sequence:
[SEQ ID NO: 5]
QTPSSLSASLGDRVTISCRASQDIKNYLNWYQQPGGTVKVLIYYTSRLHS
GVPSRFSGSGSGTDYSLTISNLEQEDFATYFCQRGDTLPWTFGGGTKLEI
K (18E12 VL);
and
a VH domain that comprises, consists or consists essentially of the sequence: LESGPGLVAPSQSLSITCTVSGFSLTNYDISWIRQPPGKGLEWLGVIWTSGGTNYNSAFMSRLSI TKDNSESQVFLKMNGLQTDDTGIYYCVRGDGNFYLYNFDYWGQGTTLTVSS [SEQ ID NO: 6] (18E12 VH).

30. The method of claim 27, wherein the complement pathway inhibitor is a C5a inhibitor.

31. The method of claim 30, wherein the C5a inhibitor is PMX205 or eculizumab.

32. The method of claim 27, wherein the agent that blocks the interaction between CD40 and CD40 ligand is an antibody that binds specifically to CD40 and/or CD40 ligand.

33. The method of claim 32, wherein the antibody is AT-1502.

34. The method of any one of claims 23 to 33, wherein the treatment regimen comprises exposing the subject to non-invasive ventilation.

35. The method of claim 34, wherein the non-invasive ventilation comprises Average Volume Assured Pressure Support (AVAPS), Continuous Positive Airway Pressure (CPAP) and/or Bilevel Positive Airway Pressure (BiPAP).

36. The method of claim 34 or 35, wherein the subject is exposed to non-invasive ventilation earlier than if the subject was likely to have slow progressing ALS.

37. A method for treating a subject that is likely to have slow progressing ALS, the method comprising:
(a) selecting a subject that is likely to have slow progressing ALS on the basis of the level of a biomarker in a biological sample of the subject, wherein the biomarker is sCD14 or LBP; and
(b) exposing the subject to a treatment regimen optimized for treating slow progressing ALS.

38. The method of claim 37, wherein (a) comprises selecting a subject that is likely to have slow progressing ALS on the basis of the levels of sCD14 and LBP in the biological sample of the subject.

39. The method of claim 37 or 38, further comprising, prior to selecting the subject, determining whether the subject is likely to have slow progressing ALS in accordance with the method of any one of claims 1-12.

40. The method of any one of claims 37 to 39, wherein the treatment regimen does not comprise the administration of an anti-neurodegenerative agent.

41. The method of any one claims 37 to 40, wherein the treatment regimen comprises the administration of an anti-neurodegenerative agent.

42. The method of claim 41, wherein the anti-neurodegenerative agent is selected from among riluzole, edaravone, a CD14 antagonist, GM604, masitinib, a complement pathway inhibitor, and an agent that blocks the interaction between CD40 and CD40 ligand.

43. The method of claim 42, wherein the CD14 antagonist is a CD14 antagonist antibody.

44. The method of claim 43, wherein the CD14 antagonist antibody is selected from:
(1) an antibody comprising:
a VL domain that comprises, consists or consists essentially of the sequence:
[SEQ ID NO: 1]
QSPASLAVSLGQRATISCRASESVDSFGNSFMHWYQQKAGQPPKSSIYRA
ANLESGIPARFSGSGSRTDFTLTINPVEADDVATYFCQQSYEDPWTFGGG
TKLGNQ (3C10 VL);
and
a VH domain that comprises, consists or consists essentially of the sequence:
[SEQ ID NO: 2]
LVKPGGSLKLSCVASGFTFSSYAMSWVRQTPEKRLEWVASISSGGTTYYP
DNVKGRFTISRDNARNILYLQMSSLRSEDTAMYYCARGYYDYHYWGQGTT
LTVSS (3C10 VH);
(2) an antibody comprising:
a VL domain that comprises, consists or consists essentially of the sequence:
[SEQ ID NO: 3]
QSPASLAVSLGQRATISCRASESVDSYVNSFLHWYQQKPGQPPKLLIYRA
SNLQSGIPARFSGSGSRTDFTLTINPVEADDVATYCCQQSNEDPTTFGGG
TKLEIK (28C5 VL);
and
a VH domain that comprises, consists or consists essentially of the sequence:
[SEQ ID NO: 4]
LQQSGPGLVKPSQSLSLTCTVTGYSITSDSAWNWIRQFPGNRLEWMGYIS
YSGSTSYNPSLKSRISITRDTSKNQFFLQLNSVTTEDTATYYCVRGLRFA
YWGQGTLVTVSA (28C5 VH);
and
(3) an antibody comprising:
a VL domain that comprises, consists or consists essentially of the sequence: QTPSSLSASLGDRVTISCRASQDIKNYLNWYQQPGGTVKVLIYYTSRLHSGVPSRFSGSGSGTDY SLTISNLEQEDFATYFCQRGDTLPWTFGGGTKLEIK [SEQ ID NO: 5] (18E12 VL); and
a VH domain that comprises, consists or consists essentially of the sequence:
[SEQ ID NO: 6]
LESGPGLVAPSQSLSITCTVSGFSLTNYDISWIRQPPGKGLEWLGVIWTS
GGTNYNSAFMSRLSITKDNSESQVFLKMNGLQTDDTGIYYCVRGDGNFYL
YNFDYWGQGTTLTVSS (18E12 VH).

45. The method of claim 42, wherein the complement pathway inhibitor is a C5a inhibitor.

46. The method of claim 45, wherein the C5a inhibitor is PMX205 or eculizumab.

47. The method of claim 42, wherein the agent that blocks the interaction between CD40 and CD40 ligand is an antibody that binds specifically to CD40 and/or CD40 ligand.

48. The method of claim 47, wherein the antibody is AT-1502.

49. The method of any one of claims 23 to 48, wherein the biological sample is selected from the group consisting of blood, plasma, serum, urine and CSF.

50. Use of an antigen-binding molecule specific for a biomarker in the preparation of a kit for determining whether a subject with ALS is likely to have rapid or slow progressing ALS or for assessing the rate of progression of ALS in a subject, wherein the biomarker is sCD14 or LBP.

51. The use of claim 50, wherein the use is of the combination of an antigen-binding molecule specific for sCD14 and an antigen-binding molecule specific for LBP in the preparation of a kit for determining whether a subject with ALS is likely to have rapid or slow progressing ALS or for assessing the rate of progression of ALS in a subject.

52. The use of claim 50 or 51, further comprising the use of an antigen-binding molecule specific for at least one other biomarker in the preparation of the kit.

53. The use of claim 52, wherein the at least one other biomarker is selected from among CRP, MIF, sTNFRI, sTNFRII, NFL, pNfH, p75NTR^{ECD}, miR-206, miR-143-3p, and miR-374b-5p.
